(19) United States
(12) Patent Application Publication
(10) Pub. No.: US 2024/0386990 A1
(43) Pub. Date: Nov. 21, 2024

(54) PREDICTING CELLULAR RESPONSES TO PERTURBATIONS

(71) Applicant: Insitro, Inc., South San Francisco, CA (US)

(72) Inventors: Theofanis Karaletsos, San Francisco, CA (US); Michael Bereket, San Carlos, CA (US)

(21) Appl. No.: 18/667,968

(22) Filed: May 17, 2024

Related U.S. Application Data
(60) Provisional application No. 63/467,577, filed on May 18, 2023.

(51) Int. Cl. G16B 5/20; C12N 9/22; C12N 15/11; C12Q 1/6841; C12Q 1/6874; G16B 40/30

(52) U.S. Cl. CPC G16B 5/20; C12N 9/22; C12N 15/11; C12Q 1/6841; C12Q 1/6874; G16B 40/30; C12N 2310/20

(57) ABSTRACT

Trained machine learning models are deployed to generate predictions of cellular responses to perturbations. A treated representation of a cell is generated within a latent space using one or more disentangled representations, examples of which include a basal state representation of a cell, a learned treatment mask for a perturbation, and/or a treatment representation for the perturbation. Within the latent space, effects of perturbations are modeled as inducing sparse latent offsets. Multiple perturbations can be modeled in the latent space as the sparse latent offsets compose additively (sparse additive mechanism shift). Thus, operating within this latent space enables the modeling of cellular responses to one or more perturbations.

### CROSS-REFERENCE TO RELATED APPLICATIONS

This application claims the benefit of and priority to U.S. Provisional Patent Application No. 63/467,577 filed May 18, 2023, the entire disclosure of which is hereby incorporated by reference in its entirety for all purposes.

### BACKGROUND OF THE INVENTION

Evaluating cellular responses to perturbations in in vitro or in vivo settings are often costly and time intensive, thereby severely limiting the extent that such experiments are conducted. Therefore, there is a need for improved methods for evaluating cellular responses to perturbations.

### SUMMARY

Disclosed herein are methods, non-transitory computer readable media, and systems for generating predictions of cellular responses to perturbations. In various embodiments, a trained machine learning model is deployed to analyze a treated representation of a cell in a latent space, thereby generating a prediction of a cellular response to a perturbation. A treated representation of a cell is generated using one or more disentangled representations in the latent space, examples of which include a basal state representation of a cell, a learned treatment mask for the perturbation, and/or a treatment representation for the perturbation. Put more generally, within the latent space, the one or more disentangled representations are used to model perturbations as inducing sparse latent offsets in the latent space. In particular embodiments, multiple perturbations can be modeled in the latent space as the sparse latent offsets compose additively (sparse additive mechanism shift). Altogether, deploying generative models and operating within this latent space enables the modeling (e.g., in silico modeling) of cellular responses to one or more perturbations.

Comparatively, previously developed models include the Compositional Perturbation Autoencoder (CPA), as described by Lotfollahi, M. et al., Learning interpretable cellular responses to complex perturbations in high-throughput screens. bioRxiv, 2021.04.14.439903, which is hereby incorporated by reference in its entirety, as well as Sparse Variational Autoencoder (SVAE+), as described by Lopez et al., Learning causal representations of single cells via sparse mechanism shift modeling, 2022, arXiv:2211.03553, which is hereby incorporated by reference in its entirety. Here, each of CPA and SVAE+ suffers drawbacks or lack of functionality that are addressed by the trained machine learning models disclosed herein (referred to herein as “Sparse Additive Mechanism Shift Variational Autoencoder” or “SAMS-VAE”). For example, SAMS-VAE comprises a generative model that explicitly models sparsity in latent perturbation effects that can compose additively. Thus, SAMS-VAE can perform modeling of two or more perturbations on individual cells that have not previously been encountered. In contrast, CPA is not a generative model and does not specify a prior for the latent basal state (therefore any predictions must start from an observed cell). CPA further does not learn sparse representations and therefore, does not allow sparsity in the perturbations. SVAE+ does not model treatment latent variables separately and does not have a mechanism to compose interventions. SVAE+ must model combinations as entirety new perturbations, thereby limiting the degree to which knowledge of individual perturbations can be transferred to combinations of perturbations.

Disclosed herein is a method of performing modeling of a cellular response to one or more perturbations, the method comprising: accessing a plurality of disentangled representations in a latent space, the plurality of disentangled representations comprising: a basal state representation (z^{b}) of a cell; a learned treatment mask (mₜ) for a perturbation; and a treatment representation (eₜ) for the perturbation; generating a sparse treatment representation (z^{p}) representing a combination of the learned treatment mask (mₜ) and the treatment representation (eₜ); combining the basal state representation (z^{b}) and the sparse treatment representation (z^{p}) to generate a treated representation (z) representing a perturbed cell in the latent space; and predicting the cellular response to the one or more perturbations using the treated representation (z) representing a perturbed cell in the latent space.

In various embodiments, predicting the cellular response comprises determining a magnitude of a shift in the latent space between the treated representation (z) representing a perturbed cell in the latent space and the basal state representation (z^{b}). In various embodiments, predicting the cellular response comprises decoding the treated representation (z) in the latent space to predict a phenotypic output of the cell in response to the one or more perturbations. In various embodiments, the phenotypic output comprises one or more of cell sequencing data, protein expression data, gene expression data, image data, cell metabolic data, cell morphology data, or cell interaction data. In various embodiments, the gene expression data comprises RNA-seq data. In various embodiments, the RNA-seq data comprises single-cell RNA-seq data or bulk RNA-seq data. In various embodiments, the cell sequencing data comprises in situ RNA sequencing data.

In various embodiments, decoding the treated representation (z) in the latent space comprises applying a likelihood function. In various embodiments, the likelihood function comprises a negative binomial likelihood model or a Gaussian likelihood model. In various embodiments, the likelihood function is parameterized by a learned inversion dispersion parameter θ_{g}. In various embodiments, the disentangled representations in the latent space comprise embeddings. In various embodiments, the basal state representation (z^{b}) of the cell comprises a vector embedding comprising values sampled from a learned distribution. In various embodiments, the treatment representation (eₜ) for the perturbation comprises a vector embedding comprising values sampled from a learned distribution. In various embodiments, the learned treatment mask (mₜ) for the perturbation comprises sparse values. In various embodiments, the sparse values of the learned treatment mask (mₜ) reflect locations in the latent space affected by the perturbation. In various embodiments, methods disclosed herein further comprise using the sparse values of the learned treatment mask (mₜ) to identify one or more affected cellular pathways in response to the perturbation.

In various embodiments, the perturbation is one of a chemical agent, molecular intervention, environmental mimic, or genetic editing agent. In various embodiments, the genetic editing agent comprises a CRISPR system. In various embodiments, the plurality of disentangled representations in the latent space are generated using phenotypic assay data captured from one or more cells. In various embodiments, the phenotypic assay data comprises one or more of cell sequencing data, protein expression data, gene expression data, image data, cell metabolic data, cell morphology data, or cell interaction data. In various embodiments, the phenotypic assay data are captured from one or more non-perturbed cells. In various embodiments, the phenotypic assay data are captured from one or more cells that have been exposed to one or more perturbations. In various embodiments, the phenotypic assay data are captured from one or more non-perturbed cells and further captured from one or more cells that have been exposed to one or more perturbations. In various embodiments, the phenotypic assay data are captured from one or more in vitro cells.

In various embodiments, the plurality of disentangled representations in the latent space are generated by: training an autoencoder comprising: an encoder configured to analyze a phenotypic output of a cell to generate one or more corresponding representations in the latent space; and a decoder configured to analyze a representation in the latent space to generate a predicted phenotypic output. In various embodiments, training the autoencoder comprises jointly training the encoder and the decoder. In various embodiments, training the autoencoder further comprises: disentangling a representation generated by the encoder in the latent space into a basal state representation of a cell and a treatment representation for a perturbation. In various embodiments, training the autoencoder further comprises: training a distribution for sampling a learned treatment mask (mₜ) for a perturbation, the learned treatment mask (mₜ) comprising sparse values reflecting affected cellular pathways in the latent space in response to the perturbation. In various embodiments, training an autoencoder comprises training the autoencoder using RNA expression data. In various embodiments, the RNA expression data comprises single-cell RNA-seq data or bulk RNA-seq data. In various embodiments, training an autoencoder comprises training the autoencoder using Perturb-seq data. In various embodiments, the Perturb-seq data is generated by providing one or more of a small molecule drug, a biologic, or a genetic perturbation to in vitro cells. In various embodiments, training an autoencoder comprises training the autoencoder using cell images comprising phenotypic outputs of cells. In various embodiments, the cell images are captured by performing in situ sequencing by synthesis through a pooled optical screening. In various embodiments, the method achieves a R2 performance metric for differentially expressed genes of at least 0.72. In various embodiments, the method achieves an importance weighted evidence lower bound (IWELBO) performance metric of less than −1756.00.

In various embodiments, methods disclosed herein further comprise: identifying a target biomarker using the predicted cellular response to the one or more perturbations. In various embodiments, identifying the target biomarker comprises correlating the predicted cellular response to one or more sparse values of the learned treatment mask (mₜ) for the perturbation. In various embodiments, identifying the target biomarker further comprises accessing one or more additional learned treatment masks (mₜ) for one or more additional perturbations, the one or more additional learned treatment masks (mₜ) also including the one or more sparse values. In various embodiments, identifying the target biomarker comprises determining the target biomarker corresponding to the one or more sparse values of the learned treatment mask and the additional learned treatment masks. In various embodiments, the plurality of disentangled representations further comprise: a second learned treatment mask (mₜ_{₂}) for a second perturbation; and a second treatment representation (eₜ_{₂}) for the second perturbation. In various embodiments, methods disclosed herein further comprise: generating a second sparse treatment representation (z^{p}^{₂}) representing a combination of the second learned treatment mask (mₜ_{₂}) and the second treatment representation (eₜ_{₂}). In various embodiments, methods disclosed herein further comprise: combining the combination of the basal state representation (z^{b}) and the sparse treatment representation (z^{p}) with the second sparse treatment representation (z^{p}^{₂}) to generate the treated representation (z). In various embodiments, predicting the cellular response to the one or more perturbation by deploying the trained model comprises predicting the cellular response to the perturbation and the second perturbation.

In various embodiments, methods disclosed herein further comprise: performing a reversion screen using at least the predicted cellular response to the one or more perturbations. In various embodiments, performing the reversion screen comprises: determining whether effects of the second perturbation revert effects of the first perturbation, wherein the effects of the first perturbation comprise a latent shift of the combination of the basal state representation (z^{b}) and the sparse treatment representation (z^{p}), and wherein the effects of the second perturbation comprise a latent shift of the combination of the second sparse treatment representation (z^{p}^{₂}) with the combination of the basal state representation (z^{b}) and the sparse treatment representation (z^{p}). In various embodiments, the method is performed in silico.

Additionally disclosed herein is a method of performing modeling of a cellular response to a first perturbation and a second perturbation, the method comprising: accessing a plurality of disentangled representations in a latent space, the plurality of disentangled representations comprising: a basal state representation (z^{b}) of a cell; a first learned treatment mask (mₜ_{₁}) for the first perturbation; a second learned treatment mask (mₜ_{₂}) for the second perturbation; a first treatment representation (eₜ_{₁}) for the first perturbation; a second treatment representation (eₜ_{₂}) for the second perturbation; generating a first sparse treatment representation (z^{p}^{₁}) representing a combination of the first learned treatment mask (mₜ_{₁}) and the first treatment representation (eₜ_{₁}); generating a second sparse treatment representation (z^{p}^{₂}) representing a combination of the second learned treatment mask (mₜ_{₂}) and the second treatment representation (eₜ_{₂}); combining the basal state representation (z^{b}) and the first sparse treatment representation (z^{p}^{₁}) and the second sparse treatment representation (z^{p}^{₂}) to generate a treated representation (z) representing a perturbed cell in the latent space; and predicting the cellular response to the perturbation using the treated representation (z) representing a perturbed cell in the latent space.

Additionally disclosed herein is a method of performing a reversion screen, the method comprising: accessing a plurality of disentangled representations in a latent space, the plurality of disentangled representations comprising: a basal state representation (z^{b}) of a cell; a first learned treatment mask (mₜ_{₁}) for a first perturbation; a second learned treatment mask (mₜ_{₂}) for a second perturbation; a first treatment representation (eₜ_{₁}) for the first perturbation; a second treatment representation (eₜ_{₂}) for the second perturbation; generating a first sparse treatment representation (z^{p}^{₁}) representing a combination of the first learned treatment mask (mₜ_{₁}) and the first treatment representation (eₜ_{₁}); generating a second sparse treatment representation (z^{p}^{₂}) representing a combination of the second learned treatment mask (mₜ_{₂}) and the second treatment representation (eₜ_{₂}); comparing the first sparse treatment representation (z^{p}^{₁}) and the second sparse treatment representation (z^{p}^{₂}) to determine whether effects of the second perturbation counteract effects of the first perturbation. In various embodiments, the effects of the first perturbation comprise a latent shift imparted by the first sparse treatment representation (z^{p}^{₁}), and wherein the effects of the second perturbation comprise a latent shift of the second sparse treatment representation (z^{p}^{₂}). In various embodiments, comparing the first sparse treatment representation (z^{p}^{₁}) and the second sparse treatment representation (z^{p}^{₂}) further comprises: combining the basal state representation (z^{b}) and the first sparse treatment representation (z^{p}^{₁}) and the second sparse treatment representation (z^{p}^{₂}) to generate a treated representation (z) representing a perturbed cell in the latent space; and predicting the cellular response to the perturbation using the treated representation (z) representing a perturbed cell in the latent space.

Additionally disclosed herein is a method of performing modeling of a cellular response to one or more perturbations, the method comprising: obtaining or having obtained a treated representation (z) representing a perturbed cell in a latent space, the treated representation (z) generated by combining a basal state representation (z^{b}) of the cell and a sparse treatment representation (z^{p}), the sparse treatment representation (z^{p}) representing a combination of a learned treatment mask (mₜ) for a perturbation and a treatment representation (eₜ) for the perturbation; and predicting the cellular response to the one or more perturbations by deploying a trained model to analyze the treated representation (z) representing a perturbed cell in the latent space. In various embodiments, predicting the cellular response comprises deploying the trained model to decode the treated representation (z) in the latent space to predict a phenotypic output of the cell in response to the one or more perturbations. In various embodiments, the phenotypic output comprises one or more of cell sequencing data, protein expression data, gene expression data, image data, cell metabolic data, cell morphology data, or cell interaction data. In various embodiments, the gene expression data comprises RNA-seq data. In various embodiments, the RNA-seq data comprises single-cell RNA-seq data or bulk RNA-seq data. In various embodiments, the cell sequencing data comprises in situ RNA sequencing data. In various embodiments, decoding the treated representation (z) in the latent space comprises applying a likelihood function. In various embodiments, the likelihood function comprises a negative binomial likelihood model or a Gaussian likelihood model. In various embodiments, the likelihood function is parameterized by a learned inversion dispersion parameter θ_{g}. In various embodiments, one or more of the basal state representation (z^{b}) of a cell, the learned treatment mask (mₜ) for the perturbation, and treatment representation (eₜ) for the perturbation are embeddings. In various embodiments, one or more of the basal state representation (z^{b}) of a cell, the learned treatment mask (mₜ) for the perturbation, and treatment representation (eₜ) for the perturbation are neural network embeddings. In various embodiments, the basal state representation (z^{b}) of the cell comprises a vector embedding comprising values sampled from a learned distribution. In various embodiments, the treatment representation (eₜ) for the perturbation comprises a vector embedding comprising values sampled from a learned distribution. In various embodiments, the learned treatment mask (mₜ) for the perturbation comprises sparse values. In various embodiments, the sparse values of the learned treatment mask (mₜ) reflect locations in the latent space affected by the perturbation.

In various embodiments, methods disclosed herein further comprise using the sparse values of the learned treatment mask (mₜ) to identify one or more affected cellular pathways in response to the one or more perturbations. In various embodiments, the one or more perturbations are one of a chemical agent, molecular intervention, environmental mimic, or genetic editing agent. In various embodiments, the genetic editing agent comprises a CRISPR system. In various embodiments, the plurality of disentangled representations in the latent space are generated using phenotypic assay data captured from one or more cells. In various embodiments, the phenotypic assay data comprises one or more of cell sequencing data, protein expression data, gene expression data, image data, cell metabolic data, cell morphology data, or cell interaction data. In various embodiments, the phenotypic assay data are captured from one or more non-perturbed cells. In various embodiments, the phenotypic assay data are captured from one or more cells that have been exposed to one or more perturbations. In various embodiments, the phenotypic assay data are captured from one or more non-perturbed cells and further captured from one or more cells that have been exposed to one or more perturbations. In various embodiments, the phenotypic assay data are captured from one or more in vitro cells.

In various embodiments, one or more of the basal state representation (z^{b}) of a cell, the learned treatment mask (mₜ) for the perturbation, and treatment representation (eₜ) for the perturbation are generated by: training an autoencoder comprising: an encoder configured to analyze a phenotypic output of a cell to generate one or more corresponding representations in the latent space; and a decoder configured to analyze a representation in the latent space to generate a predicted phenotypic output. In various embodiments, training the autoencoder comprises jointly training the encoder and the decoder. In various embodiments, training the autoencoder further comprises: disentangling a representation generated by the encoder in the latent space into a basal state representation of a cell and a treatment representation for a perturbation. In various embodiments, training the autoencoder further comprises: training a distribution for sampling a learned treatment mask (mₜ) for a perturbation, the learned treatment mask (mₜ) comprising sparse values reflecting affected cellular pathways in the latent space in response to the one or more perturbations. In various embodiments, training an autoencoder comprises training the autoencoder using RNA expression data. In various embodiments, the RNA expression data comprises single-cell RNA-seq data or bulk RNA-seq data. In various embodiments, training an autoencoder comprises training the autoencoder using Perturb-seq data. In various embodiments, the Perturb-seq data is generated by providing one or more of a small molecule drug, a biologic, or a genetic perturbation to in vitro cells. In various embodiments, training an autoencoder comprises training the autoencoder using cell images comprising phenotypic outputs of cells. In various embodiments, the cell images are captured by performing in situ sequencing by synthesis through a pooled optical screening. In various embodiments, the method achieves a R2 performance metric for differentially expressed genes of at least 0.72. In various embodiments, the method achieves an importance weighted evidence lower bound (IWELBO) performance metric of less than −1756.00.

In various embodiments, methods disclosed herein further comprise: identifying a target biomarker using the predicted cellular response to the one or more perturbations. In various embodiments, identifying the target biomarker comprises correlating the predicted cellular response to one or more sparse values of the learned treatment mask (mₜ) for the perturbation. In various embodiments, identifying the target biomarker further comprises accessing one or more additional learned treatment masks (mₜ) for one or more additional perturbations, the one or more additional learned treatment masks (mₜ) also including the one or more sparse values. In various embodiments, identifying the target biomarker comprises determining the target biomarker corresponding to the one or more sparse values of the learned treatment mask and the additional learned treatment masks.

In various embodiments, the plurality of disentangled representations further comprise: a second learned treatment mask (mₜ_{₂}) for a second perturbation; and a second treatment representation (eₜ_{₂}) for the second perturbation. In various embodiments, the treated representation (z) is further generated by combining the basal state representation (z^{b}) of the cell and the sparse treatment representation (z^{p}) of the perturbation with a second sparse treatment representation (z^{p}^{₂}) for a second perturbation. In various embodiments, the second sparse treatment representation (z^{p}^{₂}) represents a combination of a second learned treatment mask (mₜ_{₂}) and a second treatment representation (eₜ_{₂}) for the second perturbation. In various embodiments, predicting the cellular response to the one or more perturbation by deploying the trained model comprises predicting the cellular response to the perturbation and the second perturbation.

In various embodiments, methods disclosed herein further comprise: performing a reversion screen using at least the predicted cellular response to the one or more perturbations. In various embodiments, performing the reversion screen comprises: determining whether effects of the second perturbation revert effects of the first perturbation, wherein the effects of the first perturbation comprise a latent shift of the combination of the basal state representation (z^{b}) and the sparse treatment representation (z^{p}), and wherein the effects of the second perturbation comprise a latent shift of the combination of the second sparse treatment representation (z^{p}^{₂}) with the combination of the basal state representation (z^{b}) and the sparse treatment representation (z^{p}). In various embodiments, the method is performed in silico.

Additionally disclosed herein is a method of performing modeling of a cellular response to a first perturbation and a second perturbation, the method comprising: obtaining or having obtained a treated representation (z) representing a perturbed cell exposed to the first perturbation and the second perturbation in a latent space, the treated representation (z) generated by combining a basal state representation (z^{b}) of the cell and a first sparse treatment representation (z^{p}^{₁}) for the first perturbation and a second sparse treatment representation (z^{p}^{₂}) for the second perturbation, wherein the first sparse treatment representation (z^{p}^{₁}) represents a combination of a first learned treatment mask (mₜ_{₁}) for the first perturbation and a first treatment representation (eₜ_{₁}) for the first perturbation, wherein the second sparse treatment representation (z^{p}^{₂}) represents a combination of a second learned treatment mask (mₜ_{₂}) and a second treatment representation (eₜ_{₂}) for the second perturbation; and predicting the cellular response to the first perturbation and the second perturbation by deploying a trained model to analyze the treated representation (z) representing a perturbed cell in the latent space.

Additionally disclosed herein is a non-transitory computer readable medium comprising instructions that, when executed by a processor, cause the processor to perform any of the methods disclosed herein. Additionally disclosed herein is a system comprising: a processor; and a non-transitory computer readable medium comprising instructions that, when executed by the processor, cause the processor to perform any of the methods disclosed herein.

### BRIEF DESCRIPTION OF THE DRAWINGS

These and other features, aspects, and advantages of the present invention will become better understood with regard to the following description and accompanying drawings. It is noted that, wherever practicable, similar or like reference numbers may be used in the figures and may indicate similar or like functionality. For example, a letter after a reference numeral, such as “third party entity 740A,” indicates that the text refers specifically to the element having that particular reference numeral. A reference numeral in the text without a following letter, such as “third party entity 740,” refers to any or all of the elements in the figures bearing that reference numeral (e.g. “third party entity 740” in the text refers to reference numerals “third party entity 740A” and/or “third party entity 740B” in the figures).

FIG. 1 depicts an overall system environment involving an in silico cell perturbation system, in accordance with an embodiment.

FIG. 2 depicts an example block diagram of the in silico cell perturbation system, in accordance with an embodiment.

FIG. 3 depicts an example flow diagram for deploying a machine learning model to generate a prediction of a cellular response to one or more perturbations, in accordance with an embodiment.

FIG. 4A depicts an example flow diagram for generating a treated representation (z) of a cell in the latent space, in accordance with a first embodiment.

FIGS. 4B and 4C show an example process for generating a treated representation (z) of a cell in the latent space, in accordance with the first embodiment.

FIG. 4D depicts an example flow diagram for generating a treated representation (z) of a cell in the latent space, in accordance with a second embodiment.

FIG. 5A depicts an example flow diagram for training a machine learning model, in accordance with an embodiment.

FIG. 5B depicts an example reversion screen, in accordance with an embodiment.

FIG. 6 depicts an example flow process for predicting a cellular response to a perturbation, in accordance with an embodiment.

FIG. 7A illustrates an example computing device for implementing the system and methods described in FIGS. 1-3, 4A-4D, 5, and 6A.

FIG. 7B depicts an overall system environment for implementing an in silico cell perturbation system, in accordance with an embodiment.

FIG. 7C is an example depiction of a distributed computing system environment for implementing the system environment of FIG. 7B.

FIG. 8 depicts an example generative model for predicting a cellular response to a perturbation.

FIGS. 9A-9C show example treatment embeddings for the SAMS-VAE, SVAE+, and CPA-VAE methodologies.

FIG. 10 depicts an example learned treatment mask for the SAMS-VAE methodology.

FIGS. 11A-11B depict an example visualization of latent mask and embeddings across perturbations, sorted by hierarchical clustering.

FIGS. 12A-12B depict an example scatter plot of inferred mask probabilities and expected embedding value for inferred perturbation embeddings.

FIG. 13A shows example UMAP clusters of SAMS-VAE latent treatment embeddings and overlapping pathway annotations.

FIG. 13B shows hierarchical clustering of inferred treatment embeddings.

FIG. 14 shows a heat map of correlations between latent dimensions and gene expression values.

FIG. 15 is an example scatter plot of average treatment effects of each CRISPR guide on the expression of each gene. Effects on genes directly targeted by guides are colored orange.

FIGS. 16A and 16B show test set IWELBO results from norman-ood and norman-data-efficiency experiments. IWELBO values are plotted relative to the mean IWELBO of SAMS-VAE models with 0 combinations for each held-out split.

FIG. 17 depicts an example SAMS-VAE model that incorporates covariates.

FIGS. 18A-18F show exemplary embeddings demonstrating the applicability of SAMS-VAE on pooled optical screening data.

### DETAILED DESCRIPTION OF THE INVENTION

### Definitions

Terms used in the claims and specification are defined as set forth below unless otherwise specified.

The phrase “disentangled representations” refers to representations modeling different aspects that are independent of each other. Example disentangled representations include a basal state representation of a cell, a treatment representation of a perturbation, or a learned treatment mask for a perturbation.

The phrases “basal state representation” or “z^{b}” are used interchangeably and refer to a representation of a cell within the latent space. In particular embodiments, the basal state representation is a representation of the component of a cell's state not due to perturbation within the latent space. In various embodiments, the basal state representation is a vector embedding of a cell in the latent space.

The phrases “treatment representation” or “eₜ” are used interchangeably and refer to a representation of a perturbation within the latent space. A representation of a perturbation within the latent space encompasses a representation of effects of the perturbation on a cell within the latent space. In various embodiments, the treatment representation is a vector embedding of a perturbation in the latent space.

The phrases “learned treatment mask” or “mₜ” are used interchangeably and refer to a latent representation that performs a feature selection. For example, the learned treatment mask for the perturbation, when combined with a treatment representation, performs a feature selection to retain certain values while eliminating other values of the treatment representation. In various embodiments, the learned treatment mask is a sparse binary treatment mask. Such a sparse binary treatment mask includes values of “0” and “1”, where a value of “1” indicates a feature that is to be retained and a value of “0” indicates a feature that is to be eliminated.

The phrase “sparse binary mask” refers to a learned treatment mask for a perturbation including binary values (e.g., values of “0” and “1”) in which at least 50% of the values of the sparse binary mask are “0”. In various embodiments, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the values of the sparse binary mask are “0”.

The phrase “phenotypic assay data” includes any data that provides information about a cell phenotype, such as, e.g., cell sequencing data (e.g., RNA sequencing data, sequencing data related to epigenetics such as methylation state), protein expression data, gene expression data, image data (e.g., high-resolution microscopy data or immunohistochemistry data), cell metabolic data, cell morphology data, or cell interaction data. In various embodiments, phenotypic assay data refers to cell sequencing data. In particular embodiments, phenotypic assay data refers to RNA sequencing data (RNA-seq data). In particular embodiments, phenotypic assay data refers to image data (e.g., microscopy data).

The phrase “obtaining or having obtained a treated representation (z) representing a perturbed cell” involves obtaining the treated representation (z) in a latent space. The phrase further encompasses accessing the treated representation (z) in a latent space and/or generating the treated representation (z) in the latent space. The phrase also encompasses receiving the treated representation (z) e.g., from a third party that has performed the steps to generate the treated representation (z).

The phrase “in silico” refers to a methodology performed on a computer or via computer simulation. In some embodiments, in silico methods are exclusively performed on a computer or via computer simulation without non-in silico methods (e.g., in vitro or in vivo methods). In various embodiments, in silico methods are performed on a computer or via computer simulation in addition to non-in silico methods (e.g., in vitro or in vivo methods).

It must be noted that, as used in the specification and the appended claims, the singular forms “a,” “an” and “the” include plural referents unless the context clearly dictates otherwise.

### Overview of System Environment

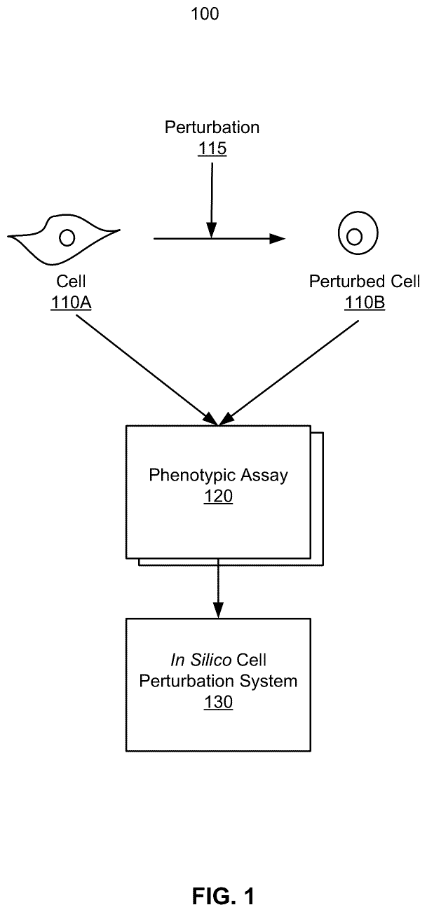

FIG. 1 depicts an overall system environment 100 involving an in silico cell perturbation system 130, in accordance with an embodiment. As shown in FIG. 1, the overall system environment 100 further includes one or more phenotypic assays 120 performed on cell 110A and/or perturbed cell 110B. Here, cell 110A may be exposed to a perturbation 115, which induces the cell 110A into a perturbed cell 110B in a perturbed state.

In various embodiments, the cell 110A (and correspondingly the perturbed cell 1101B) represents any of a single cell, a population of cells, or multiple populations of cells. Populations of cells can vary in regards to the type of cells (single cell type, mixture of cell types), cell lineage (e.g., cells in differing stages of maturation or differing stages of disease progression), or cell culture (e.g., in vivo, in vitro 2D culture, in vitro 3D culture, or in vitro organoid or organ-on-chip systems). In various embodiments, the cell 110A (and correspondingly the perturbed cell 1101B) is a somatic cell. In various embodiments, the cell 110A (and correspondingly the perturbed cell 110B) is a differentiated cell. In various embodiments, the cell 110A (and correspondingly the perturbed cell 1101B) is a differentiated cell. In various embodiments, the cell 110A (and correspondingly the perturbed cell 1101B) is differentiated from a primary cell (e.g., transdifferentiated). In various embodiments, the cell 110A (and correspondingly the perturbed cell 110B) is differentiated from a stem cell. In various embodiments, the cell 110A (and correspondingly the perturbed cell 1101B) is differentiated from an induced pluripotent stem cell (iPSCs). In particular embodiments, the cell 110A (and correspondingly the perturbed cell 1101B) is a neuronal cell. In particular embodiments, the cell 110A (and correspondingly the perturbed cell 1101B) is a microglia. In particular embodiments, the cell 110A (and correspondingly the perturbed cell 110B) is an astrocyte. In particular embodiments, the cell 110A (and correspondingly the perturbed cell 110B) is an oligodendrocyte. In particular embodiments, the cell 110A (and correspondingly the perturbed cell 110B) is a hepatocyte cell. In particular embodiments, the cell 110A (and correspondingly the perturbed cell 110B) is a hepatic stellate cell (HSC). In particular embodiments, the cell 110A (and correspondingly the perturbed cell 110B) is a K562 cell. In particular embodiments, the cell 110A (and correspondingly the perturbed cell 110B) is a A549 cell. In particular embodiments, the cell 110A (and correspondingly the perturbed cell 110B) is a neuronal iPSC cell.

In various embodiments, the cell 110A includes a population of cells with varying genetic backgrounds. For example, a first cell of the population may have a genetic background that differs from at least another cell of the population. Genetic background covers the underlying genetics of a cell, such as the underlying genomic DNA sequence of the cell. In various embodiments, a genetic background of a cell includes one or more genetic changes such as mutations (e.g., polymorphisms, single nucleotide polymorphisms (SNPs), single nucleotide variants (SNVs)), insertions, deletions, knock-ins, knock-outs). In various embodiments, a population of cells may include genetic backgrounds that mimic a patient or a population of patients (e.g., cells of the population have similar genetic backgrounds as the patients).

In various embodiments, a perturbation 115 induces a cell 110A into a state of disease relevant for a clinical endpoint of interest. Therefore, exposing a cell 110A to a perturbation 115 results in an altered (e.g., non-normal or non-healthy) state in the perturbed cell 110B. Example perturbations, such as chemical agents, molecular interventions, environmental mimics, or genetic editing agents are described herein. As a specific example, a perturbation may be a TGFβ perturbation which induces a cell 110A into a diseased state of fibrosis exhibited by a perturbed cell 110B. In various embodiments, various perturbations can be provided to different cells 110A to generate different perturbed cells 110B that are reflective of different clinical endpoints of interest and/or different diseases. Here, the various perturbations can be useful for generating vast quantities of phenotypic assay data, such as exposure response phenotypes, that are useful for training robust machine learning models. Further details of exposure response phenotypes are described in U.S. application Ser. No. 17/350,761, which is incorporated by reference in its entirety.

In various embodiments, the perturbation of cells 110A is performed in an array format. For example, cells 110A are individually plated (e.g., in separate wells) and individually perturbed. In some embodiments, the perturbation of cells is performed in a pooled format. For example, cells 110A are pooled together and perturbed. In one embodiment, the pooled cells are exposed to the same perturbation. In one embodiment, the cells in the pool are individually exposed to individual perturbations.

In various embodiments, cells are exposed to varying doses of a perturbation 115. For example, a first cell may be exposed to a first dose of a perturbation 115 and a second cell may be exposed to a second dose of a perturbation 115. Here, the first dose and second dose may differ (e.g., first dose may be less than or greater than the second dose). In various embodiments, different cells may be exposed to two, three, four, five, six, seven, eight, nine, or ten or more doses. Example doses of a perturbation 115 include e.g., any of 0.1 ng/mL, 0.2 ng/mL, 0.3 ng/mL, 0.4 ng/mL, 0.5 ng/mL, 0.6 ng/mL, 0.7 ng/mL, 0.8 ng/mL, 0.9 ng/mL, 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 15 ng/mL, 20 ng/mL, 25 ng/mL, 30 ng/mL, 35 ng/mL, 40 ng/mL, 45 ng/mL, 50 ng/mL, 60 ng/mL, 70 ng/mL, 75 ng/mL, 80 ng/mL, 90 ng/mL, 100 ng/mL, 150 ng/mL, 200 ng/mL, 250 ng/mL, 300 ng/mL, 350 ng/mL, 400 ng/mL, 450 ng/mL, 500 ng/mL, 600 ng/mL, 700 ng/mL, 800 ng/mL, 900 ng/mL, 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 15 μg/mL, 20 μg/mL, 30 μg/mL, 40 μg/mL, 50 μg/mL, 60 μg/mL, 70 μg/mL, 80 μg/mL, 90 μg/mL, 100 μg/mL, 150 μg/mL, 200 μg/mL, 250 μg/mL, 300 μg/mL, 350 μg/mL, 400 μg/mL, 450 μg/mL, 500 μg/mL, 550 μg/mL, 600 μg/mL, 700 μg/mL, 800 μg/mL, 900 μg/mL, or 1 mg/mL.

Generally, one or more phenotypic assays 120 are performed to capture phenotypic assay data from the cell 110A and/or perturbed cell 110B. Phenotypic assay data generally refers to any data that provides information about a cell phenotype. In various embodiments, phenotypic assay data captured from a cell 110A can be reflective of a non-diseased state of the cell 110A whereas phenotypic assay data captured from perturbed cell 110B can be reflective of a diseased state of the perturbed cell 110B.

In various embodiments, phenotypic assay data refers to cell sequencing data. In particular embodiments, phenotypic assay data refers to RNA sequencing data (RNA-seq data). In particular embodiments, phenotypic assay data refers to image data (e.g., microscopy data). In various embodiments, phenotypic assay data refers to both cell sequencing data (e.g., RNA-seq data) as well as image data (e.g., microscopy data). Further details of example phenotypic assays as well as phenotypic assay data are described herein.

As shown in FIG. 1, phenotypic assay data from the one or more phenotypic assays 120 are provided to the in silico cell perturbation system 130. In various embodiments, the in silico cell perturbation system 130 trains machine learning models using the phenotypic assay data. In various embodiments, the in silico cell perturbation system 130 deploys machine learning models for generating in silico predictions of cellular responses to perturbations.

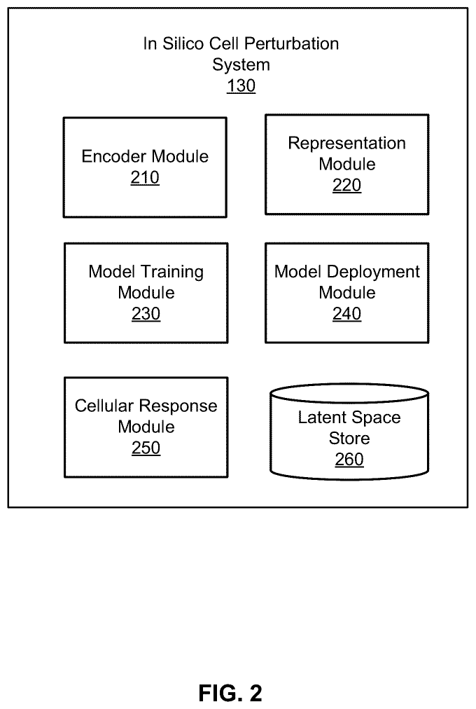

Reference is now made to FIG. 2, which depicts an example block diagram of the in silico cell perturbation system, in accordance with an embodiment. FIG. 2 introduces individual components of the in silico cell perturbation system 130, examples of which include an encoder module 210, a representation module 220, model training module 230, a model deployment module 240, a cellular response module 250, and a latent space store 260.

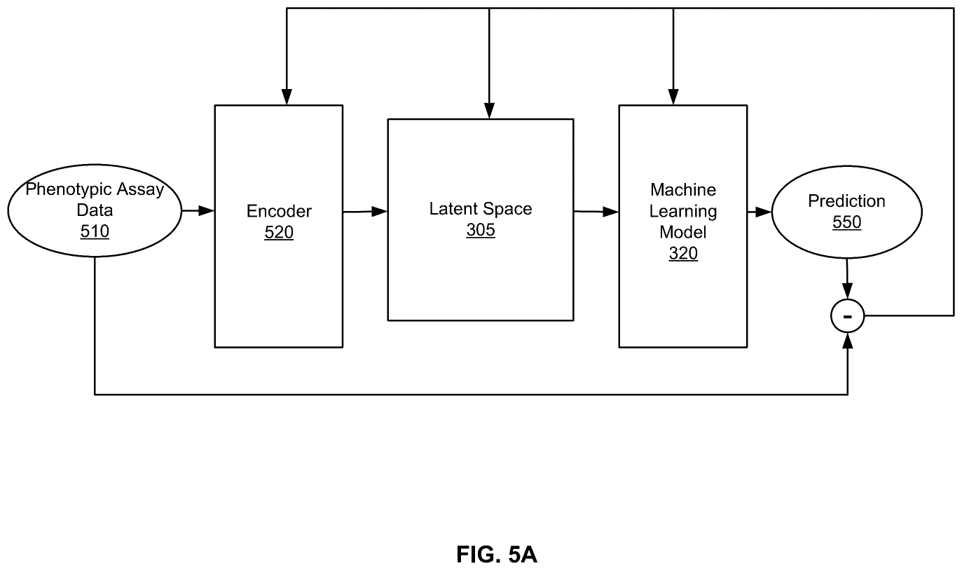

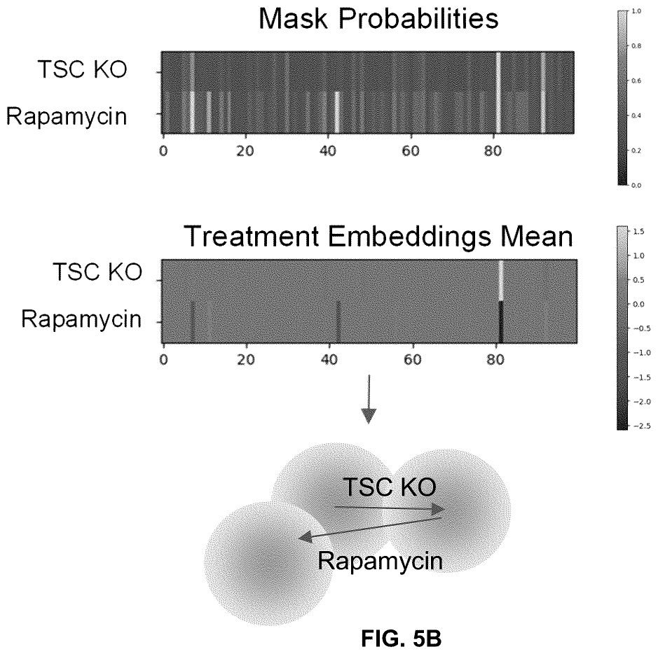

Referring to the encoder module 210, it transforms the phenotypic assay data captured from cells into representations of a latent space. In various embodiments, the encoder module 210 applies an encoder that transforms the phenotypic assay data captured from cells. In various embodiments, the encoder is a sub-component of an autoencoder (as is further described in reference to FIG. 5). In various embodiments, the encoder is a machine learned model, such as a neural network. For example, the encoder may be a feedforward, densely connected network. Thus, by applying the encoder, the encoder module 210 transforms the phenotypic assay data into a latent space representation. In particular embodiments, the latent space representation of the phenotypic assay data has reduced dimensionality in comparison to the phenotypic assay data itself. In various embodiments, the encoder need not explicitly be a machine learned model. For example, the encoder module 210 may perform a multiple probability simulation, an example of which may involve a Monte Carlo method, to transform the phenotypic assay data into a latent space representation.

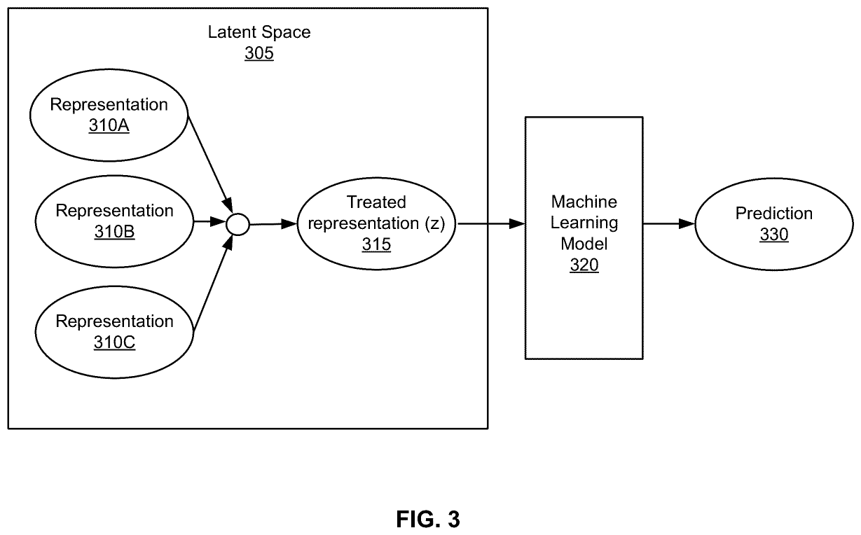

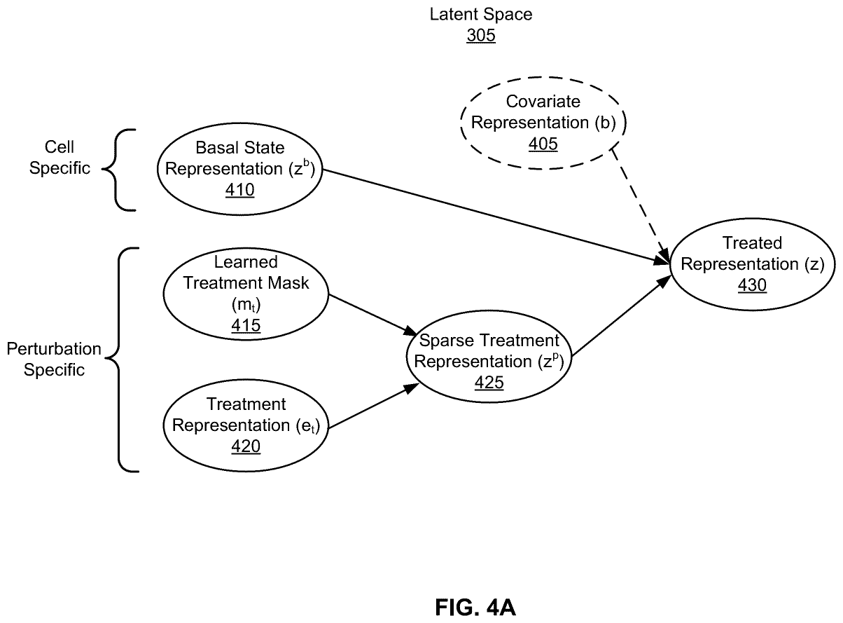

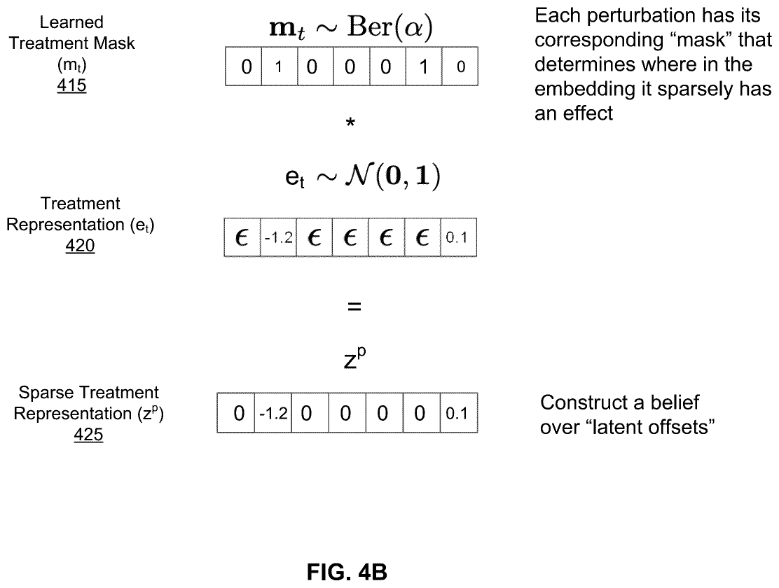

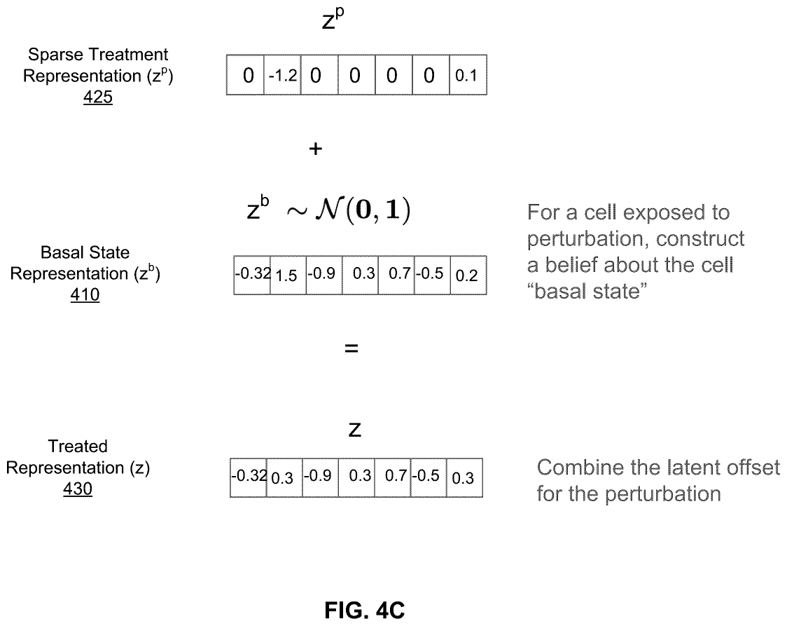

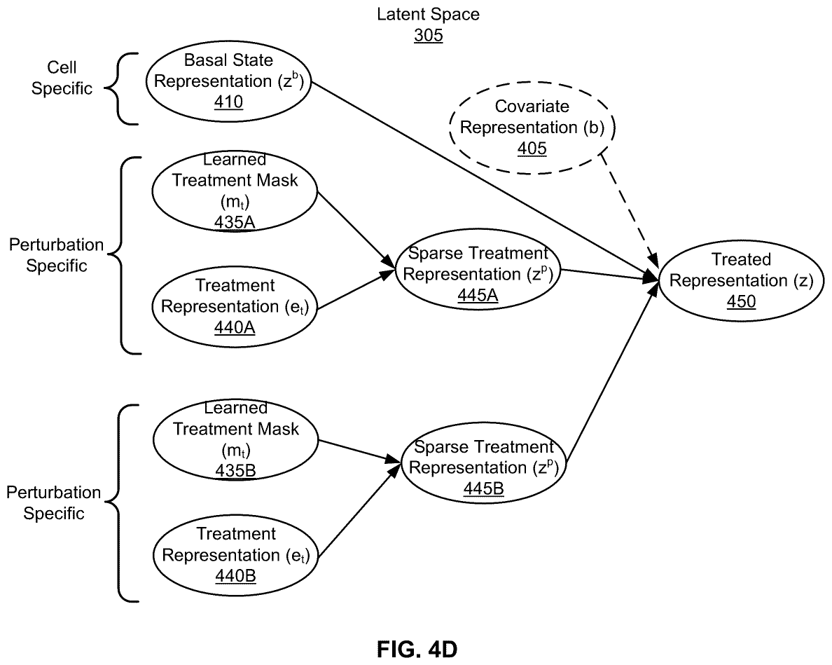

Referring to the representation module 220, it processes representations in the latent space. In various embodiments, the representation module 220 accesses disentangled representations in the latent space and combines disentangled representations to generate a treated representation (z). Here, the treated representation (z) represents a perturbed cell (e.g., a perturbed cell exposed to one or more perturbations) in the latent space. Thus, the treated representation (z) can be used to generate in silico predictions of cellular responses to perturbations. Further details of steps performed by the representation module 220 are described herein in reference to at least FIGS. 3 and 4A-4D.

Referring to the model training module 230, it trains machine learning models using a training dataset. In various embodiments, the training dataset includes phenotypic assay data captured from cells (e.g., cell 110A and/or perturbed cell 110B) as described herein. Thus, the model training module 230 may train machine learning models using at least phenotypic assay data captured from cells. In various embodiments, the machine learning model is a sub-component of an autoencoder. For example, the machine learning model may be a decoder of an autoencoder. In various embodiments, the model training module 230 trains the machine learning model of the autoencoder by jointly training the sub-components of the autoencoder. For example, the model training module 230 may jointly train an encoder and a decoder of the autoencoder. In various embodiments, an autoencoder architecture need not be employed and therefore, the model training module 230 may train machine learning models (without further training an encoder).

In various embodiments, the model training module 230 further generates one or more learned distributions in the latent space. For example, learned distributions in the latent space can be used for accessing disentangled representations. For example, a learned distribution may be learned distributions useful for accessing basal state representations (z^{b}), learned treatment masks (mₜ), and/or treatment representations (eₜ). Further details of steps performed by the model training module 230 are described herein in reference to at least FIG. 5.

Referring to the model deployment module 240, it deploys trained machine learning models to generate predictions of cellular responses to perturbations. In various embodiments, such predictions of cellular responses to perturbations are in silico predictions (e.g., predictions for a cell that are performed completely in silico). Generally, the model deployment module 240 deploys trained machine learning models to analyze treated representations (z) of perturbed cells in the latent space. Here, a treated representation (z) of a perturbed cell incorporates sparse latent offsets in the latent space that arise due to effects of one or more perturbations. The trained machine learning models analyze treated representations (z) of a perturbed cell and output a prediction, an example of which includes phenotypic outputs corresponding to the perturbed cell. In various embodiments, a trained machine learning model decodes a treated representation (z) in the latent space to predict phenotypic outputs of the cell in response to the perturbation. Further details of the processes performed by the model deployment module 240 are described herein.

In various embodiments, the cellular response module 250 determines a cellular response of a cell based on at least the outputted prediction by a trained machine learning model. In various embodiments, the outputted prediction from a trained machine learning model represents phenotypic outputs corresponding to a perturbed cell or the outputted prediction can be used to determine a predicted phenotypic output corresponding to the perturbed cell. In various embodiments, the phenotypic output includes one or more of cell sequencing data, protein expression data, gene expression data, image data, cell metabolic data, cell morphology data, or cell interaction data. In particular embodiments, the phenotypic output includes cell sequencing data or gene expression data, which includes RNA-seq data. In various embodiments, the RNA-seq data comprises single-cell RNA-seq data or bulk RNA-seq data. In various embodiments, the cell sequencing data includes in situ RNA sequencing data.

In various embodiments, the cellular response module 250 predicts a cellular response to one or more perturbations. For example, the cellular response module 250 may determine a magnitude of a shift in the latent space between the treated representation (z) representing a perturbed cell in the latent space and a basal state representation (z^{b}). In various embodiments, the larger the shift in the latent space, the larger the cellular response to the one or more perturbations. For example, for a first cell, the shift in latent space between the treated representation (z) representing a perturbed first cell in the latent space and a basal state representation (z^{b}) of the first cell may be significant, thereby indicating that the first cell exhibits a significant cellular response due to the one or more perturbations. For a second cell, the shift in latent space between the treated representation (z) representing the perturbed second cell in the latent space and a basal state representation (z^{b}) of the second cell may not be significant, thereby indicating that the second cell exhibits a minimal cellular response due to the one or more perturbations. In some embodiments, the magnitude of the shift in the latent space does not correspond to the magnitude of a cellular response.

Generally, the predictions of cellular responses to perturbations are useful for performing large-scale virtual compounds screens. For example, large number of perturbations (e.g., hundreds, thousands, millions, or even billions) can be screened in silico to understand their likely impact on cellular responses. Furthermore, such perturbations can be combined in silico to model their combined effects on cells. It would be more time-consuming and resource intensive to screen such large numbers of perturbations (and combinations of these perturbations) through current in vitro or in vivo screens. An additional benefit is that in silico predictions of cellular responses to perturbations can be generated across various types of cells and/or cells with different genetic backgrounds. Therefore, different cellular responses to perturbations that may arise due to the different cell types or due to the different genetic backgrounds can be uncovered through large-scale in silico compound screens.

In various embodiments, the predictions of cellular responses to perturbations are useful for performing reversion screens. Using the methods disclosed herein, a large number of perturbations can be screened to identify perturbations or combinations of perturbations that would cause a cell to revert from a first state to a second state. In various embodiments, the first state is a diseased state. In various embodiments, the second state is a non-diseased or a less diseased state. As a specific example, a first perturbation may be modeled to induce a cell to move from a basal state into a diseased state. Then, the effects of a second perturbation can be modeled in silico to determine the extent to which the second perturbation influences the reversion of the cell into a less diseased or a non-diseased state. Here, the effects of the first perturbation and the second perturbation can be composed additively, thereby revealing the individual effects of each perturbation as well as the overall effects of all perturbations.

### Example Methods for Deploying Machine Learning Models for Predicting Cellular Response to Perturbations

As described herein, methods involve deploying trained machine learning models for predicting cellular responses to perturbations. In various embodiments, methods involve accessing a plurality of disentangled representations in a latent space, the plurality of disentangled representations comprising: a basal state representation (z^{b}) of a cell; a learned treatment mask (mₜ) for the perturbation; and a treatment representation (eₜ) for the perturbation; generating a sparse treatment representation (z^{p}) representing a combination of the learned treatment mask (mₜ) and the treatment representation (eₜ); combining the basal state representation (z^{b}) and the sparse treatment representation (z^{p}) to generate a treated representation (z) representing a perturbed cell in the latent space; and predicting the cellular response to the perturbation using the treated representation (z) representing a perturbed cell in the latent space.

In various embodiments, methods involve obtaining or having obtained a treated representation (z) representing a perturbed cell in a latent space, the treated representation (z) generated by combining a basal state representation (z^{b}) of the cell and a sparse treatment representation (z^{p}), the sparse treatment representation (z^{p}) representing a combination of a learned treatment mask (mₜ) for the perturbation and a treatment representation (eₜ) for the perturbation; and predicting the cellular response to the perturbation by deploying a trained model to analyze the treated representation (z) representing a perturbed cell in the latent space.

Reference is made to FIG. 3, which depicts an example flow diagram for deploying a machine learning model to generate a prediction of a cellular response to one or more perturbations, in accordance with an embodiment. Here, FIG. 3 depicts three different representations (e.g., representation 310A, representation 310B, and representation 310C) that are obtained in the latent space 305. In various embodiments, additional or fewer representations 310 may be obtained in the latent space 305. In some embodiments, two representations are obtained in the latent space 305. In some embodiments, four representations, five representations, six representations, seven representations, eight representations, nine representations, or ten representations are obtained in the latent space 305. In various embodiments, a representation in the latent space 305 is an embedding. In various embodiments, each representation in the latent space is an embedding.

In various embodiments, a representation 310 in the latent space 305 includes a basal state representation (z^{b}) of a cell (e.g., a cell that is modeled in silico to predict cellular responses to a perturbation). In various embodiments, a representation 310 in the latent space 305 includes a learned treatment mask (mₜ) for a perturbation. In various embodiments, a representation 310 in the latent space 305 includes a treatment representation (eₜ) for the perturbation. In various embodiments, a first representation (e.g., representation 310A) includes a basal state representation (z^{b}) of a cell (e.g., a cell that is modeled in silico to predict cellular responses to a perturbation), and a second representation (e.g., representation 310B) includes a treatment representation (eₜ) for the perturbation. In various embodiments, a first representation (e.g., representation 310A) includes a basal state representation (z^{b}) of a cell (e.g., a cell that is modeled in silico to predict cellular responses to a perturbation), and a second representation (e.g., representation 310B) includes a treatment representation (eₜ) for the perturbation, and a third representation includes a learned treatment mask (mₜ) for the perturbation. Here, the three representations (e.g., basal state representation (z^{b}) of a cell, treatment representation (eₜ) for the perturbation, and learned treatment mask (mₜ) for the perturbation) are useful for generating an in silico prediction of cellular response to a single perturbation.

In various embodiments, methods involve generating in silico predictions of cellular response to two perturbations. In such embodiments, methods may involve obtaining five or more representations. For example, a first representation may be a basal state representation (z^{b}) of a cell (e.g., a cell that is modeled in silico to predict cellular responses to a perturbation). The second and third representations may include a treatment representation (eₜ_{₁}) for a first perturbation and a learned treatment mask (mₜ_{₁}) for the first perturbation, respectively. The fourth and fifth representations may include a treatment representation (eₜ_{₂}) for a second perturbation and a learned treatment mask (mₜ_{₂}) for the second perturbation, respectively. In additional embodiments, methods involve generating in silico predictions of cellular to response to more than two perturbations. Each representation corresponds to two representations (e.g., a treatment representation, and a learned treatment mask). Therefore, when modeling effects of X perturbations, methods involve obtaining 2X+1 representations (the one extra representation being the basal state representation of the cell).

In various embodiments, the representations 310 in the latent space 305 represent disentangled representations. As used herein, “disentangled representations” refer to representations modeling different aspects that are disentangled (e.g., independent) of each other. For example, a first disentangled representation may include a basal state representation (z^{b}) of a cell, which is a baseline representation in the latent space of a cell. A second disentangled representation may include a treatment representation (eₜ) for a perturbation, which is a representation of the perturbation in the latent space. Here, the basal state representation (z^{b}) of a cell and the treatment representation (eₜ) for a perturbation model different aspects of the cell and the perturbation, respectively. Thus, the basal state representation (z^{b}) of a cell and the treatment representation (eₜ) for a perturbation are individually different disentangled representations.

In some embodiments, a disentangled representation is a learned treatment mask (mₜ) for the perturbation. In various embodiments, the learned treatment mask (mₜ) for the perturbation represents a feature selection process that emphasizes particular features of the perturbation in the latent space. In various embodiments, the basal state representation (z^{b}) of a cell, the treatment representation (eₜ) for a perturbation, and the learned treatment mask (mₜ) for the perturbation are individually different disentangled representations. Generally, a learned treatment mask (mₜ) for the perturbation can be indicative of biological underpinnings that are reflective of cellular changes in response to perturbations. The learned treatment mask (mₜ) learns latent dimensions and/or latent features that respond to the perturbation. Here, example latent features can be abstractions of cellular processes, cellular pathways, biomarkers (e.g., biomarkers useful for characterizing cells or for targeting or modulating for purposes of treatment), and/or other phenotypic features of cellular change.

In various embodiments, obtaining a representation, such as any of a basal state representation (z^{b}) of a cell, a treatment representation (eₜ) for a perturbation, or a learned treatment mask (mₜ) for a perturbation, involves sampling a learned distribution. A learned distribution can be any of a Bernoulli distribution, a Normal distribution, a binomial distribution, a negative binomial distribution, a beta-binomial distribution, a Poisson distribution, a hypergeometric distribution, a negative hypergeometric distribution, or a Rademacher distribution. In particular embodiments, a learned distribution is a Bernoulli distribution. In particular embodiments, a learned distribution is a Normal distribution. As described herein, a learned distribution may be parameterized with a parameter ϕ. In various embodiments, a learned distribution is parameterized with a variational distribution parameter ϕ.

One advantage of the generative model described here in FIG. 3 is that predictions of cellular responses can be generated for not-previously encountered cells. For example, the basal state representation (z^{b}) of a cell can be sampled from a learned distribution. Therefore, the basal state representation (z^{b}) can be a representation of a not-previously encountered cell. This is in contrast to non-generative models (e.g., CPA) which is limited to modeling only previously observed cells.

In various embodiments, a representation can be expressed as a vector embedding, examples of which are described herein (e.g., with respect to FIGS. 4B and 4C). Thus, the values of the vector embedding can be sampled from a distribution. In various embodiments, the distribution is a prior distribution. For example, any of the basal state representation (z^{b}) of a cell, the treatment representation (eₜ) for a perturbation, and/or the learned treatment mask (mₜ) for a perturbation can be sampled from a prior distribution. In various embodiments, a prior distribution may be a prior distribution over continuous space (as opposed to latent space). In various embodiments, the prior distribution is a Normal distribution or a Bernoulli distribution, such as a multi-dimensional Bernoulli distribution. In various embodiments, a representation can be sampled from a learned posterior distribution. For example, a posterior distribution may be a distribution learned during training to replace the prior distribution. The posterior distribution can be a learnable distribution of arbitrarily complex shape. In various embodiments, for the basal state representation (z^{b}) of a cell and the treatment representation (eₜ) for a perturbation, they are sampled from learned posterior distributions over continuous values. In various embodiments for the learned treatment mask (mₜ) for a perturbation, it is sampled from a learned posterior distribution over binary values.

In various embodiments, the values of a basal state representation (z^{b}) of a cell can be sampled from a learned distribution trained or generated at least from training data captured from cells in a basal state (e.g., non-perturbed state). In particular embodiments, the learned distribution trained or generated at least from training data captured from cells in a basal state comprises a Normal distribution. As one example, the values of a treatment representation (eₜ) for a perturbation can be sampled from a learned distribution trained or generated at least from training data captured from perturbed cells. In particular embodiments, the learned distribution trained or generated at least from training data captured from perturbed cells comprises a Normal distribution. As one example, the values of a learned treatment mask (mₜ) for a perturbation can be sampled from a learned distribution trained or generated at least from training data captured from perturbed cells. In particular embodiments, the learned distribution trained or generated at least from training data captured from perturbed cells comprises a Bernoulli distribution.

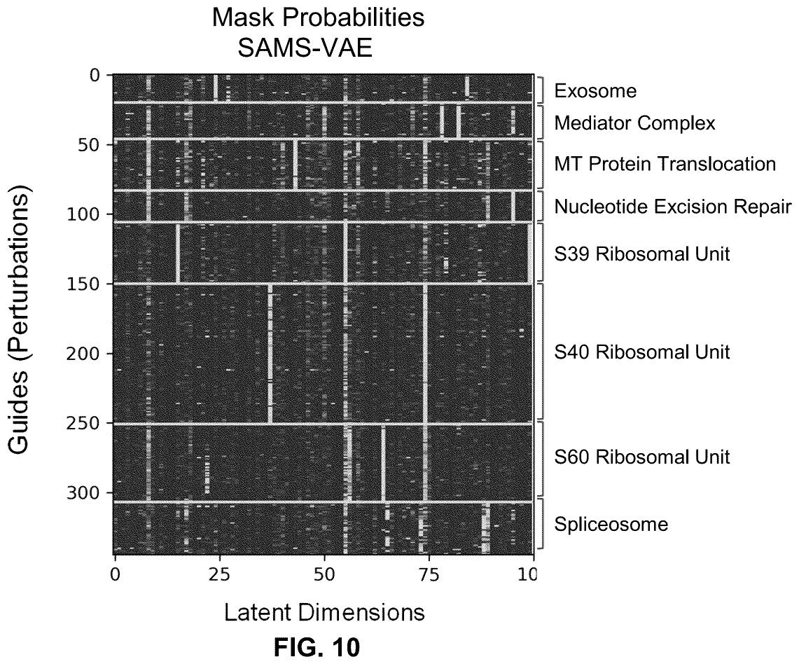

In particular embodiments, the learned treatment mask (mₜ) for a perturbation comprises sparse values. In such embodiments, the learned treatment mask (mₜ) is a sparse binary treatment mask, which includes values of “0” and “1”. In various embodiments, the sparse values of the learned treatment mask (mₜ) reflect locations in the latent space affected by the perturbation. For example, a value of “1” in the learned treatment mask (mₜ) indicates a feature that is to be retained and a value of “0” in the learned treatment mask (mₜ) indicates a feature that is to be eliminated. In various embodiments, the sparse values of the learned treatment mask (mₜ) are indicative of biological underpinnings that are reflective of cellular changes in response to perturbations. For example, the sparse values of the learned treatment mask (mₜ) can be useful for identifying one or more affected cellular pathways in response to the perturbation. Example learned treatment masks (mₜ) are described herein in reference to FIG. 10, which shows alignment of the values of the learned treatment masks (mₜ) relative to categorized cellular pathways.

Returning to FIG. 3, the flow diagram involves combining the representations 310 to generate a treated representation (z) in the latent space 305. Here, the treated representation (z) represents a perturbed cell in the latent space 305. In various embodiments, the treated representation (z) models the effects on a cell due to one or more perturbations in the latent space. In particular embodiments, the effects of the one or more perturbations are modeled as offsets in the latent space (e.g., shifts within the latent space). For example, if one or more perturbations cause significant cellular responses, then in the latent space, the one or more perturbations would cause a significant shift in the latent space. In other words, the treated representation (z) of the perturbed cell would be located in the latent space far from a location in the latent space of the basal state representation (z^{b}) of the cell. Comparatively, if one or more perturbations do not cause significant cellular responses, then the one or more perturbations would result in a less significant shift in the latent space. In other words, the treated representation (z) of the perturbed cell would be located in the latent space close to a location in the latent space of the basal state representation (z^{b}) of the cell.

In various embodiments, a decoding step is performed to decode the treated representation (z) 315. In various embodiments, the decoding step involves applying a machine learning model 320, as shown in FIG. 3. For example, the machine learning model 320 analyzes the treated representation (z) 315 and generates a prediction 330. In various embodiments, the machine learning model 320 decodes the treated representation (z) from the latent space into the prediction 330. In various embodiments, the prediction 330 may represent a phenotypic output, such as any of cell sequencing data, protein expression data, gene expression data, image data, cell metabolic data, cell morphology data, or cell interaction data. In particular embodiments, a phenotypic output refers to RNA-seq data. Given that the treated representation (z) represents a perturbed cell, the RNA-seq data can be useful for determining expression of genes of the perturbed cell. In various embodiments, the RNA-seq data comprises single-cell RNA-seq data or bulk RNA-seq data. In various embodiments, the prediction 330 is a predicted effect size of a perturbation given a particular cell phenotypic output. In various embodiments, the prediction 330 is a predicted size of a perturbation among 2 or more perturbations given a particular cell phenotypic output.

In various embodiments, the decoding step involves applying a likelihood function. In various embodiments, the machine learning model 320 can generate a prediction 330 useful for determining a probability of an observation (e.g., an observation of a phenotypic output) using the likelihood function. For example, the phenotypic output may be RNA-seq data therefore, the prediction 330 may be useful for determining a probability of an observation in the RNA-seq data, such as the increase or decrease in expression of a particular gene. In various embodiments, the prediction 330 represents a predicted parameter that parameterizes a likelihood function. In various embodiments, the predicted parameter is an inversion dispersion parameter, hereafter referred to as θ_{g}. The likelihood function provides a probability of an observation (e.g., an observation of a phenotypic output). In various embodiments, the likelihood function is one of a negative binomial likelihood model or a Gaussian likelihood model.

In particular embodiments, the decoding step involves applying a machine learning model 320 and further involves applying a likelihood function. In particular embodiments, the machine learning model 320 is a neural network that analyzes the treated representation (z) 315 and generates a prediction of an inversion dispersion parameter θ_{g}. The inversion dispersion parameter θ_{g} parameterizes one of a negative binomial likelihood model or a Gaussian likelihood model.

In various embodiments, the output of the likelihood model is an observation of a phenotypic output (e.g., an observation of RNA-seq or cell imaging data). For example, an observation of a phenotypic output can be sampled from a conditional likelihood p(xᵢ|z; θ) where parameters of the likelihood model are computed from the treated representation (z) using a neural network with parameters θ.

In various embodiments, the machine learning model is evaluated according to a performance metric, examples of which include an area under the curve (AUC) of a receiver operating curve, a positive predictive value, a negative predictive value, an accuracy, a correlation metric (e.g., R² performance metric), or an importance weighted evidence lower bound (IWELBO) performance metric.

In various embodiments, machine learning models disclosed herein achieve an AUC value of at least 0.5. In various embodiments, machine learning models disclosed herein achieve an AUC value of at least 0.6. In various embodiments, machine learning models disclosed herein achieve an AUC value of at least 0.7. In various embodiments, machine learning models disclosed herein achieve an AUC value of at least 0.8. In various embodiments, machine learning models disclosed herein achieve an AUC value of at least 0.9. In various embodiments, machine learning models disclosed herein achieve an AUC value of at least 0.95. In various embodiments, machine learning models disclosed herein achieve an AUC value of at least 0.99. In various embodiments, machine learning models disclosed herein achieve an AUC value of at least 0.51, at least 0.52, at least 0.53, at least 0.54, at least 0.55, at least 0.56, at least 0.57, at least 0.58, at least 0.59, at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99.

In various embodiments, machine learning models disclosed herein achieve a R² performance metric value. In various embodiments, the R² performance metric refers to a correlation between the predicted cellular response to a perturbation and differentially expressed genes. In various embodiments, machine learning models disclosed herein achieve a R² performance metric of at least 0.7. In various embodiments, machine learning models disclosed herein achieve a R² performance metric of at least 0.8. In various embodiments, machine learning models disclosed herein achieve a R² performance metric of at least 0.9. In various embodiments, machine learning models disclosed herein achieve a R² performance metric of at least 0.95. In various embodiments, machine learning models disclosed herein achieve a R² performance metric of at least 0.99. In various embodiments, machine learning models disclosed herein achieve a R² performance metric of at least 0.51, at least 0.52, at least 0.53, at least 0.54, at least 0.55, at least 0.56, at least 0.57, at least 0.58, at least 0.59, at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99.

In various embodiments, machine learning models disclosed herein achieve an importance weighted evidence lower bound (IWELBO) performance metric of less than −1800.00, less than −1790.00, less than −1780.00, less than −1770.00, less than −1760.00, or less than −1750.00. In various embodiments, machine learning models disclosed herein achieve an importance weighted evidence lower bound (IWELBO) performance metric of less than −1765.00, less than −1764.00, less than −1763.00, less than −1762.00, less than −1761.00, less than −1760.00, less than −1759.00, less than −1758.00, less than −1757.00, less than −1756.00, less than −1755.00, less than −1754.00, less than −1753.00, less than −1752.00, less than −1751.00, or less than −1750.00. The IWELBO performance metric can be measured based on a particular dataset, examples of which include the replogle-filtered or replogle-essential datasets disclosed in Replogle, J. et al., Mapping information-rich genotype-phenotype landscapes with genome-scale perturb-seq. Cell, 185(14):2559-2575.e28, 2022, which is hereby incorporated by reference in its entirety.

### Modeling Cellular Response to a Single Perturbation

FIG. 4A depicts an example flow diagram for generating a treated representation (z) of a cell in the latent space, in accordance with a first embodiment. As shown in FIG. 4A, three representations are obtained from the latent space 305. The representations include a basal state representation (z^{b}) 410, a learned treatment mask (mₜ) 415, and a treatment representation (eₜ) 420. Here, the basal state representation (z^{b}) is specific for a cell, whereas the learned treatment mask (mₜ) 415 and the treatment representation (eₜ) are specific for the perturbation. In various embodiments, the learned treatment mask (mₜ) is a sparse binary mask. Thus, when the learned treatment mask (mₜ) 415 and the treatment representation (eₜ) 420 are combined, the sparse binary values of the learned treatment mask (mₜ) select for features of the treatment representation (eₜ) 420, thereby generating a sparse treatment representation (z^{p}) 425 which retains informative features while eliminating less informative features from the treatment representation (eₜ) 420. In various embodiments, methods further involve obtaining a covariate representation (b) 405. A covariate representation (b) can be representative of impact from a covariate (e.g., an experimental setting). As an example, in the context of pooled optical screening (POSH), a covariate may include a location of a well in a well plate or a barcode identifier for the plate. As shown in FIG. 4A, the covariate representation (b) 405 may be optional (as indicated by the dotted line) and need not be present in all embodiments.

Next, the sparse treatment representation (z^{p}) is combined with the basal state representation (z^{b}) to generate the treated representation (z) 430. As described herein, the combination of the sparse treatment representation (z^{p}) and the basal state representation (z^{b}) models the effects on a cell in response to the single perturbation in the latent space 305. Here, the effects of the perturbation is modeled as an offset (e.g., shift) in the latent space.

Reference is now made to FIGS. 4B and 4C, which show an example process for generating a treated representation (z) of a cell in the latent space, in accordance with the first embodiment. FIGS. 4B and 4C show an exemplary process and is not intended to be limiting. FIG. 4B shows an exemplary learned treatment mask (mₜ) 415. Here, the learned treatment mask (mₜ) 415 is specific for a perturbation and is a sparse binary mask. Specifically, the learned treatment mask (mₜ) 415 shown in FIG. 4B is a vector including seven entries with binary values (e.g., 0 or 1). The second entry and the sixth entry in the mask have values of 1 whereas the first, third, fourth, fifth, and seventh entries in the mask have values of 0. These values of the learned treatment mask (mₜ) 415 may be sampled from a distribution, such as a Bernoulli distribution. The distribution may be trained or generated during the training phase, as is further described herein.

FIG. 4B further shows an exemplary treatment representation (eₜ) 420. Here, the treatment representation (eₜ) 420 is specific for the perturbation and is shown as a vector including seven entries. The values of the treatment representation (eₜ) 420 may be sampled from a distribution, such as a Normal distribution (e.g., N(0,1)). The values for the second entry and sixth entry are shown (although one skilled in the art would recognize that values for the other entries may also be present).

FIG. 4B further shows an exemplary sparse treatment representation (z^{p}) 425. Here, the sparse treatment representation (z^{p}) represents the latent offset of the perturbation. Given that the learned treatment mask (mₜ) 415 includes values of “1” in the second entry and the sixth entry, the sparse treatment representation (z^{p}) 425 includes values in the corresponding second entry and sixth entry. The remaining values in the other entries are masked. Thus, the learned treatment mask (mₜ) 415 operates as a feature selection process that retains informative values while eliminating less informative features from the resulting sparse treatment representation (z^{p}) 425.

Referring next to FIG. 4C, it begins with the sparse treatment representation (z^{p}) 425. FIG. 4C further shows a basal state representation (z^{b}) 410 of the cell which is depicted as a vector including seven entries. Here, the values of the basal state representation (z^{b}) 410 can be sampled from a distribution, such as a Normal distribution (e.g., N(0,1)).

FIG. 4C further shows the treated representation (z) 430. In this embodiment, the treated representation (z) 430 is the sum of the sparse treatment representation (z^{p}) 425 and the basal state representation (z^{b}) 410 of the cell. Thus, the sparse treatment representation (z^{p}) 425 shifts the basal state representation (z^{b}) 410 of the cell within the latent space to generate the treated representation (z) 430.

### Modeling Cellular Response to Two Perturbations

FIG. 4D depicts an example flow diagram for generating a treated representation (z) of a cell in the latent space, in accordance with a second embodiment. Here, FIG. 4D shows the flow diagram for modeling cellular response to two perturbations.

As shown in FIG. 4D, five representations are obtained from the latent space 305. The representations include: 1) a basal state representation (z^{b}) 410, 2) a learned treatment mask (mₜ) 435A for a first perturbation, 3) a treatment representation (eₜ) 440A for the first perturbation, 4) a learned treatment mask (mₜ) 440A for a second perturbation, and 5) a treatment representation (eₜ) 440B for the second perturbation. Here, the basal state representation (z^{b}) is specific for a cell, the learned treatment mask (mₜ) 435A and the treatment representation (eₜ) 440A are specific for a first perturbation, and the learned treatment mask (mₜ) 435B and the treatment representation (eₜ) 440B are specific for a second perturbation. In various embodiments, methods further involve obtaining a covariate representation (b) 405 that is representative of impact from a covariate (e.g., an experimental setting). As an example, in the context of pooled optical screening (POSH), a covariate may include a location of a well in a well plate or a barcode identifier for the plate. As shown in FIG. 4D, the covariate representation (b) 405 may be optional (as indicated by the dotted line) and need not be present in all embodiments.

In various embodiments, each of the learned treatment mask (mₜ) 435A and the learned treatment mask (mₜ) 435B is a sparse binary mask. Thus, when the learned treatment mask (mₜ) 435A and the treatment representation (eₜ) 440A are combined, the sparse binary values of the learned treatment mask (mₜ) 435A select for features of the treatment representation (eₜ) 440A for the first perturbation, thereby generating a sparse treatment representation (z^{p}) 445A which retains informative features of the first perturbation while eliminating less informative features from the treatment representation (eₜ) 440A. Similarly, when the learned treatment mask (mₜ) 435B and the treatment representation (eₜ) 440B are combined, the sparse binary values of the learned treatment mask (mₜ) 435B select for features of the treatment representation (eₜ) 440B for the second perturbation, thereby generating a sparse treatment representation (z^{p}) 445B which retains informative features of the second perturbation while eliminating less informative features from the treatment representation (eₜ) 440B.

Next, the basal state representation (z^{b}) 410 of the cell is combined with the sparse treatment representation (z^{p}) 445A of the first perturbation and the sparse treatment representation (z^{p}) 445B of the second perturbation to generate the treated representation (z) 430. Here, the latent offset of the first perturbation and the latent offset of the second perturbation compose additively. Given that the latent offsets are sparse, compositions of the latent offsets move disjoint subspaces, instead of composing multiple dense shifts.

### Example Machine Learning Models

Embodiments disclosed herein involve training and/or deploying machine learning models for generating predictions for cellular responses to one or more perturbations. In various embodiments, machine learning models disclosed herein can be any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, attention based models, geometric neural networks, equivariant neural networks, or recurrent networks (e.g., long short-term memory networks (LSTM), bi-directional recurrent networks, deep bi-directional recurrent networks).

In particular embodiments, machine learning models disclosed herein are neural networks, such as convolutional neural networks. In various embodiments, a machine learning model is included in an autoencoder architecture. For example, a machine learning model can refer to the decoder of an autoencoder architecture. Thus, the machine learning model decodes information from the latent space. In various embodiments, an autoencoder further includes an encoder which serves to encode information into the latent space. In various embodiments, the encoder can also be embodied as a machine learning model. In particular embodiments, the encoder is a neural network, such as a convolutional neural network. In various embodiments, the encoder need not involve a machine learning model. For example, the encoder may perform a multiple probability simulation, an example of which may involve a Monte Carlo method, to transform data into a latent space representation.

In particular embodiments involving the autoencoder architecture, the encoder is a neural network that includes a first set of layers and the decoder is a neural network that includes a second set of layers. In various embodiments, the portions of the autoencoder can be differently employed during training and deployment phases. For example, during training, both the encoder and the decoder (e.g., machine learning model) of the autoencoder are trained to learn parameters that enable the decoder (e.g., machine learning model) to generate predictions of cellular responses to perturbations. During deployment, the encoder of the autoencoder need not be implemented. For example, only the machine learning model is deployed to generate the predictions of cellular responses to perturbations.

In various embodiments, machine learning models disclosed herein can be trained using a machine learning implemented method. In various embodiments, machine learning models are implemented in an autoencoder architecture and therefore, can be jointly trained with the encoder of the autoencoder. In various embodiments, machine learning models disclosed herein can be trained using any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, gradient based optimization technique, and dimensionality reduction techniques such as manifold learning, principal component analysis, factor analysis, autoencoder, and independent component analysis, or combinations thereof. In various embodiments, the machine learning model is trained using supervised learning algorithms, unsupervised learning algorithms, semi-supervised learning algorithms (e.g., partial supervision), weak supervision, transfer, multi-task learning, or any combination thereof.

In various embodiments, machine learning models and/or the encoder of an autoencoder architecture disclosed herein have one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of a neural network, support vectors in a support vector machine, and coefficients in a regression model. The model parameters are trained (e.g., adjusted) using the training data to improve the predictive power of the machine learning model. In particular embodiments, machine learning models and/or an encoder of an autoencoder architecture include neural networks. Therefore, hyperparameters, such as the number of hidden layers, are established prior to training, and the weights associated with nodes in layers of the machine learning models and/or the encoder are adjusted during training.

### Training Machine Learning Models

Embodiments disclosed herein describe the training of machine learned models that can be deployed for generating predictions of cellular responses to perturbations. In various embodiments, trained machine learning models disclosed herein represent decoders that decode information in the latent space. For example, trained machine learning models decode a representation in the latent space to generate a prediction of a phenotypic output or a prediction that is useful for determining a phenotypic output.

In various embodiments, trained machine learning models disclosed herein are incorporated as part of an autoencoder architecture. For example, the autoencoder architecture may employ an encoder that encodes information into the latent space. The encoder is configured to analyze a phenotypic output of a cell to generate one or more corresponding representations in the latent space. The autoencoder architecture further employs the trained machine learning model that decodes the representation in the latent space. The trained machine learning model is configured to analyze a representation in the latent space to generate a prediction, such as a predicted phenotypic output. In various embodiments, training the autoencoder involves jointly training the encoder and the decoder (e.g., the machine learning model). Here, jointly training the encoder and the decoder refers to jointly adjusting the parameters of the each of the encoder and the decoder. In various embodiments, the encoder need not be a machine learning model. For example, the encoder can perform multiple probability simulation, an example of which may involve a Monte Carlo method. Thus, during training, only the machine learning model (e.g., the decoder) is trained.

Generally, the training process involves training machine learning models using training data, an example of which includes phenotypic assay data (e.g., phenotypic assay data captured by performing phenotypic assay 120 shown in FIG. 1). In various embodiments, the training process involves training machine learning models using RNA expression data. In various embodiments, the training process involves training machine learning models using RNA expression data. In various embodiments, the training process involves training machine learning models using single-cell RNA-seq data or bulk RNA-seq data. In various embodiments, the training process involves training machine learning models using Perturb-seq data. In various embodiments, Perturb-seq data is generated by providing one or more of a small molecule drug, a biologic, or a genetic perturbation to in vitro cells. Example Perturb-seq datasets are described in Replogle, J. et al., Mapping information-rich genotype-phenotype landscapes with genome-scale perturb-seq. Cell, 185(14):2559-2575.e28, 2022, which is hereby incorporated by reference in its entirety. In various embodiments, the training process involves training machine learning models using cell images comprising phenotypic outputs of cells.

In various embodiments, the machine learning models are part of an autoencoder. Therefore, the training process involves training the autoencoder using phenotypic assay data (e.g., phenotypic assay data captured by performing phenotypic assay 120 shown in FIG. 1). In various embodiments, the training process involves training the autoencoder using RNA expression data. In various embodiments, the training process involves training the autoencoder using RNA expression data. In various embodiments, the training process involves training the autoencoder using single-cell RNA-seq data or bulk RNA-seq data. In various embodiments, the training process involves training the autoencoder using Perturb-seq data. In various embodiments, Perturb-seq data is generated by providing one or more of a small molecule drug, a biologic, or a genetic perturbation to in vitro cells. Example Perturb-seq datasets are described in Replogle, J. et al., Mapping information-rich genotype-phenotype landscapes with genome-scale perturb-seq. Cell, 185(14):2559-2575.e28, 2022, which is hereby incorporated by reference in its entirety. In various embodiments, the training process involves training the autoencoder using cell images.

In various embodiments, the training process involves disentangling a representation generated by the encoder in the latent space. For example, the encoder may generate a latent representation of a perturbed cell (e.g., perturbed cell 110B shown in FIG. 1) by encoding phenotypic assay data captured from the perturbed cell. Here, the training process can further involve disentangling the latent representation of a perturbed cell into a basal state representation of the cell and a treatment representation for a perturbation that was provided to the cell. Methods for disentangling latent representations are further described in Lotfollahi, M. et al., Learning interpretable cellular responses to complex perturbations in high-throughput screens. bioRxiv, 2021.04.14.439903, which is hereby incorporated by reference in its entirety.

In various embodiments, the training process involves training a basal state representation (z^{b}) of a cell. In various embodiments, the training process involves training a treatment representation (eₜ) for a perturbation. In various embodiments, the training process involves training a learned treatment mask (mₜ) for a perturbation. In various embodiments, the training process involves training a plurality of treatment representations for a plurality of perturbations (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 250, at least 300, at least 350, at least 400, at least 450, at least 500, at least 750, at least 1000, at least 2000, at least 3000, at least 4000, at least 5000, at least 10,000, at least 20,000, at least 30,000, at least 40,000, at least 50,000, or at least 100,000 perturbations). In various embodiments, the training process involves training a plurality of learned treatment masks (mₜ) for a plurality of perturbations (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 250, at least 300, at least 350, at least 400, at least 450, at least 500, at least 750, at least 1000, at least 2000, at least 3000, at least 4000, at least 5000, at least 10,000, at least 20,000, at least 30,000, at least 40,000, at least 50,000, or at least 100,000 perturbations).

In various embodiments, the training process involves training a learned distribution that can be used to generate a basal state representation (z^{b}) of a cell. In various embodiments, the training process involves training a learned distribution that can be used to generate a treatment representation (eₜ) for a perturbation. In various embodiments, the training process involves training a learned distribution that can be used to generate a learned treatment mask (mₜ) for a perturbation. In particular embodiments, the training process involves training at least a first learned distribution that can be used to generate a basal state representation (z^{b}) of a cell, a second learned distribution that can be used to generate a treatment representation (eₜ) for a perturbation, and a third learned distribution that can be used to generate a learned treatment mask (mₜ) for a perturbation. In various embodiments, the training process can further involve training one or more additional learned distributions that can be used to generate learned treatment masks (mₜ) and/or treatment representations (eₜ) for additional perturbations. In various embodiments, the learned distributions are each parameterized (e.g., parameterized with variational distribution parameters ϕ). In various embodiments, the learned distributions are learned posterior distributions. Here, learned posterior distributions can be complex distributions to replace prior distributions (e.g., prior distributions in continuous space).

In various embodiments, a learned distribution is trained through an inference process. Example inference processes include a mean-field inference scheme (an example of which is denoted as Equation (4) described herein) or a correlated inference scheme (an example of which is denoted as Equation (5) described herein). The inference process may suggest learned posterior distributions to replace prior distributions, where a learned posterior distribution is of arbitrarily complex shape. Thus, an effect of the inference method is to learn complex posterior distributions q(z_{b}, zₜ, mₜ|data).

Reference is now made to FIG. 5, which depicts an example flow diagram for training a machine learning model 320, in accordance with an embodiment. Specifically, FIG. 5 depicts a single training iteration using training data (e.g., phenotypic assay data 510). Thus, the flow diagram shown in FIG. 5 can be performed multiple times over multiple iterations to train the machine learning model 320.

In particular, FIG. 5 introduces an autoencoder architecture which includes an encoder 520 and a machine learning model 320. Generally, the autoencoder encodes the phenotypic assay data 510 and outputs a prediction 550. In various embodiments, at each iteration, the prediction 550 can be compared to a reference ground truth of the phenotypic assay data 510 to generate a value. The value, such as a loss value, can be used to train the encoder 520, latent space 305, and/or the machine learning model 320 (e.g., through back-propagation).

Referring first to the phenotypic assay data 510, it is captured from cells (e.g., cell 110A and/or perturbed cell 110B shown in FIG. 1) by performing a phenotypic assay (e.g., phenotypic assay 120 shown in FIG. 1). In various embodiments, the phenotypic assay data 510 include observed measurements that serve as reference ground truth data. In particular embodiments, observed measurements are RNA-seq data. In particular embodiments, observed measurements are cell imaging data. In various embodiments, the phenotypic assay data 510 includes one or more annotations providing context to the observed measurements. One example of an annotation includes a dosage of a perturbation that was provided to a perturbed cell. Thus, the observed measurements were captured from the perturbed cell that was exposed to that dosage of the perturbation.

As shown in FIG. 5, the encoder 520 encodes the phenotypic assay data 510 into representations in the latent space 305. Within the latent space 305, the representations may undergo disentangling and may be used to train one or more learned distributions. Learned distributions may be trained for different types of disentangled representations. For example, a learned distribution may be trained for a basal state representation (z^{b}) of a cell. As another example, a learned distribution may be trained for a learned treatment mask (mₜ) for a perturbation. As another example, a learned distribution may be trained for a treatment representation (eₜ) for a perturbation. In various embodiments, learned distributions are trained for each of a basal state representation (z^{b}) of a cell, for a learned treatment mask (mₜ) for a perturbation, and for a treatment representation (eₜ) for a perturbation. In various embodiments, the learned distributions are each parameterized (e.g., parameterized with parameter ϕ). In various embodiments, the parameters ϕ of the learned distributions represent variational distribution parameters (e.g., parameterization of distributions of latent variables).

The machine learning model 320 decodes information in the latent space 305 to generate the prediction 550. In various embodiments, the machine learning model 320 decodes a treated representation (z) (as described herein) to generate the prediction 550. As described herein, the machine learning model 320 may include parameters θ that are adjusted during training.

In various embodiments, the prediction 550 is combined with at least the reference ground truth data of the phenotypic assay data 510. For example, the prediction is combined with the reference ground truth data of the phenotypic assay data 510 by determining a difference between the prediction and the reference ground truth data of the phenotypic assay data 510. The difference represents a value, such as a loss value, that is useful for training at least the machine learning model 320. As shown in FIG. 5, the value can be backpropagated for training each of the encoder 520 and the machine learning model 320. Additionally, the value can be backpropagated to train or generate one or more learned distributions of the latent space 305. For example, the value can be backpropagated and the variational distribution parameters ϕ of the learned distributions can be adjusted accordingly.

In various embodiments, the training process involves maximizing an evidence lower bound (ELBO) metric. For example, given the model parameters θ of the machine learning model 320 and the variational distribution parameters ϕ of the learned distributions, the training process involves maximizing the ELBO metric using stochastic gradient descent. The ELBO metric can be expressed as:

$$E L B O ( ϕ ) = {E}_{{Z}^{b} , E , M \sim q ( . \vert X , D ; ϕ )} log \frac{p ( X , {Z}^{b} , M , E \vert D ; θ )}{q ( {Z}^{b} , M , E \vert X , D ; ϕ )}$$

In such embodiments, observed measurements X (e.g., RNA-seq or imaging data) and annotated perturbation dosages D can be provided as input. An estimate for the ELBO metric can be computed using the generative model and variational distribution, and a gradient step with respect to the parameters θ and ϕ can be taken to maximize the ELBO metric.

### Example Flow Process for Predicting Cellular Response to a Perturbation

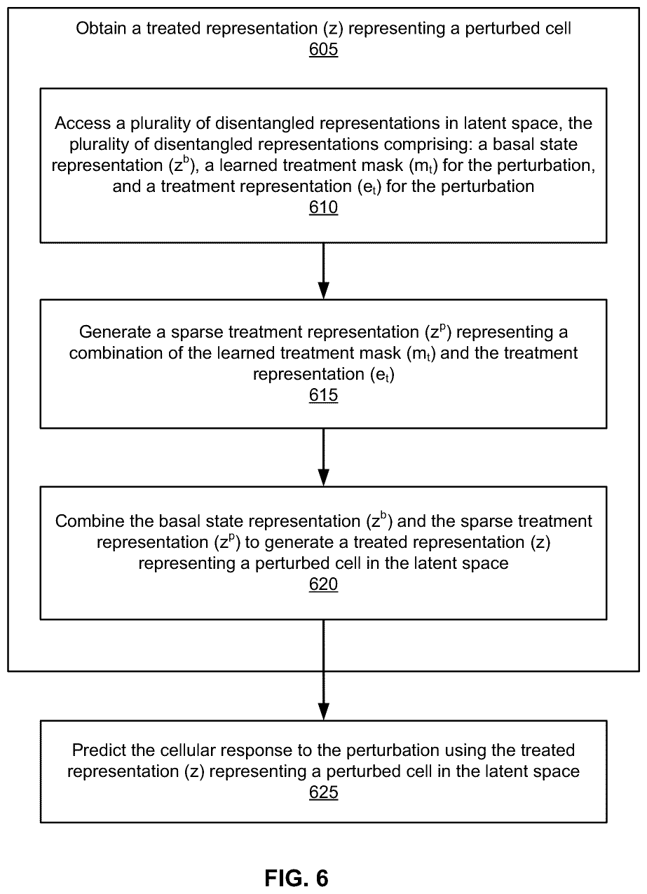

FIG. 6A depicts an example flow process for predicting a cellular response to a perturbation, in accordance with an embodiment.

Step 605 involves obtaining a treated representation z representing a perturbed cell. Here, the treated representation z is within the latent space and may represent a perturbed cell exposed to one or more perturbations. In particular embodiments, the treated representation z represents a perturbed cell exposed to two or more perturbations. In such embodiments, the treated representation z models the two or more perturbations in the latent space as additively composed latent offsets.

As shown in FIG. 6A, step 605 can involve each of steps 610, 615, and 620. Specifically, step 610 involves accessing a plurality of disentangled representations in the latent space. Here, the plurality of disentangled representations includes a basal state representation (z^{b}) of a cell, a learned treatment mask (mₜ) for the perturbation, and a treatment representation (eₜ) for the perturbation.

Step 615 involves generating a sparse treatment representation (z^{p}) representing a combination of the learned treatment mask (mₜ) and the treatment representation (eₜ). In various embodiments, the learned treatment mask (mₜ) includes learned values for the perturbation such that the sparse treatment representation (z^{p}) retains certain values while eliminating others.

Step 620 involves combining the basal state representation (z^{b}) and the sparse treatment representation (z^{p}) to generate a treated representation (z) representing a perturbed cell in the latent space. Here at step 620, given the basal state representation (z^{b}), the sparse treatment representation (z^{p}) induces a sparse latent offset within the latent space to generate the treated representation (z) of the perturbed cell.

Step 625 involves predicting the cellular response to the perturbation using the treated representation (z) representing the perturbed cell in the latent space. In various embodiments, step 625 involves deploying a trained machine learning model to analyze the treated representation (z). In various embodiments, the trained machine learning model decodes the treated representation (z) to phenotypic assay readouts (e.g., RNA-seq readouts or imaging readouts). Here, the phenotypic assay readouts are indicative of the cellular response to the perturbation.

### Additional Implementations

### Reversion Screen

In various embodiments, methods disclosed herein involve generating a prediction of cellular response to a perturbation can be useful for purposes of performing a reversion screen. As used herein, a “reversion screen” refers to a screening of a plurality of perturbations to identify a subset of perturbations that changes (e.g., reverts) a cell from a first state into a second state. In various embodiments, using the methods disclosed herein, a reversion screen can be performed for a large number of perturbations to identify perturbations that revert a cell from a diseased state into a non-diseased or a less diseased state. As a specific example, a first perturbation may be modeled to induce a cell to move from a basal state into a diseased state. Then, the effects of a second perturbation can be modeled to determine whether the second perturbation reverts the cell into a less diseased or a non-diseased state. Here, the effects of the first perturbation and the second perturbation can be composed additively, thereby revealing the individual effects of each perturbation as well as the overall effects of the perturbations.

Reference is now made to FIG. 5B, which depicts an example reversion screen, in accordance with an embodiment. Here, the reversion screen includes two perturbations. For example, a first perturbation involves a tuberous sclerosis complex (TSC) knockout (KO) (e.g., knockout imparted via a CRISPR/Cas9 system). The second perturbation involves rapamycin, a small molecule compound. Generally, TSC KO has been implicated in abnormal development and function in brain structures. Rapamycin is a therapeutic shown to normalize development in TSC suppressed brain (Cox, R., et al., Multiple Critical Periods for Rapamycin Treatment to Correct Structural Defects in Tsc-1-Suppressed Brain. Front. Mol. Neurosci. (2018), 11:409). Thus, using the methods disclosed herein, a reversion screen can be performed by modeling the combinatorial effects of TSC KO and rapamycin. FIG. 5B shows the example treatment masks (top panel of FIG. 5B) as well as example treatment embeddings (middle panel of FIG. 5B) for TSC KO and rapamycin. Notably, the treatment embeddings for both TSC KO and rapamycin include opposite effects at latent dimension 81. This indicates that the rapamycin treatment largely counteracts the effects of the TSC KO treatment. The bottom panel of FIG. 5B shows example shifts in the latent space due to the TSC KO perturbation and rapamycin. For example, within the latent space, the effects of TSC KO are modeled as a shift the rightward direction. Then, the effects of rapamycin are modeled as a shift back towards the leftward direction. There may be some additional latent effects from the rapamycin treatment, but altogether, the rapamycin reverts the cell back from a diseased state into a less diseased state.

Phenotype and/or Biomarker Discovery

As disclosed herein, methods involve training and deploying machine learning models to generate predictions of cellular responses to perturbations. For example, methods involve obtaining disentangled representations in latent space, processing the disentangled representations in the latent space, and deploying a trained machine learning model to generate the prediction of cellular response. In various embodiments, methods disclosed herein are further useful for performing phenotype and/or biomarker discovery. In particular embodiments, methods involve using one or more disentangled representations in the latent space to perform phenotype and/or biomarker discovery.

As discussed herein, the training of machine learning models can involve training one or more disentangled representations, examples of which include training a basal state representation (z^{b}) of a cell, training a treatment representation (eₜ) for a perturbation, and/or training a learned treatment mask (mₜ) for a perturbation. In various embodiments, training can include training a plurality of treatment representation (eₜ) for a plurality of perturbations and/or training a plurality of learned treatment masks (mₜ) for a plurality of perturbations. In such embodiments, phenotype and/or biomarker discovery can be performed by analyzing the plurality of treatment representation (eₜ) and/or the plurality of learned treatment masks (mₜ) of the plurality of perturbations.

In particular embodiments, phenotype and/or biomarker discovery can be performed by analyzing one or more learned treatment masks (mₜ) for one or more perturbations. In various embodiments, the sparse values of the learned treatment masks (mₜ) are indicative of biological underpinnings that are reflective of cellular changes in response to perturbations. Thus, discovery of certain phenotypes and/or biomarkers can be identified according to the sparse values of the learned treatment masks (mₜ).

Generally, the sparsity of the learned treatment mask encourages the model to learn latent representations that disentangle perturbation effects. Within the latent space, a particular dimension i can represent a learned phenotype. In contrast, without disentangled representations, it would be difficult to separate out the perturbation-relevant component of the latent space as a phenotype. If dimension i is a dimension that is consistently affected across a number of perturbations implicated in a disease, then dimension i can represent a target biomarker (e.g., for characterizing cells or for targeting as a treatment).

Reference is made to FIG. 10, which shows an example learned treatment mask (mₜ) with probability values. Specifically, the y-axis of the learned treatment mask shown in FIG. 10 shows individual perturbations. The x-axis of the learned treatment mask shown in FIG. 10 shows latent dimensions. Notably, the perturbations are grouped according to impacted cellular processes, examples of which include exosome, mediator complex, mitochondrial (MT) protein translocation, nucleotide excision repair, S39 ribosomal unit, S40 ribosomal unit, S60 ribosomal unit, and spliceosome. The perturbations are grouped according to the sparse values across the latent dimensions. For example, referring to perturbations 150-250 that are grouped in the S40 ribosomal unit cellular processes, the perturbations generally include three main sparse values (e.g., at latent dimensions of 38, 54, and 74). Thus, biomarkers corresponding to these three latent dimensions can represent promising targets (e.g., for characterizing cells or for targeting or modulating for purposes of treatment). In this example, the biomarkers corresponding to these three latent dimensions can be associated with the S40 ribosomal unit, though in other embodiments the biomarkers corresponding to these three latent dimensions need not be explicitly associated with the S40 ribosomal unit.

### Example Perturbations

Methods disclosed herein involve generating cellular responses to one or more perturbations. Generally, cellular responses are predicted by deploying trained machine learning models. In various embodiments, machine learning models are trained using phenotypic assay data captured from perturbed cells that have been exposed to one or more perturbations. For example, as described in FIG. 1, cell 110A may be exposed to a perturbation 115 to generate a perturbed cell 110B. Thus, a phenotypic assay 120 is performed to generate phenotypic assay data from the cell 110A and/or the perturbed cell 110B. Such phenotypic assay data is useful for training the machine learning models disclosed herein.

A perturbation can include an agent, such as a chemical agent, a molecular intervention, an environmental mimic, or a genetic editing agent. Examples of a genetic editing agent include a CRISPR system, such as CRISPRi or CRISPRa that serve to downregulate or overexpress certain genes, respectively. Further details regarding CRISPRi and CRISPRa and methods for transcriptional modulation using CRISPRi/a is described in U.S. application Ser. No. 15/326,428 and PCT/CN2018/117643, both which are hereby incorporated by reference in their entirety. Examples of chemical agent or a molecular intervention include genetic elements (e.g., RNA such as siRNA, shRNA, or mRNA, double or single stranded antisense oligonucleotides) as well as clinical candidates, peptides, antibodies, lipoproteins, cytokines, dietary perturbagens, metal ion salts, cholesterol crystals, free fatty acids, or A-beta aggregates. Examples of chemical agents or molecular interventions include any of CTGF/CCN2, FGF1, IFGγ, IGF1, IL1β, AdipoRon, PDGF-D, TGFβ, TNFα, HLD, LDL, VLDL, fructose, lipoic acid, sodium citrate, ACC1i (Firsocostat), ASK1i (Selonsertib), FXRa (obeticholic acid), PPAR agonist (elafibranor), CuCl₂, FeSO₄ 7H₂O, ZnSO₄ 7H₂O, LPS, TGFβ antagonist, and ursodeoxycholic acid.

In various embodiments, the perturbation is an environmental mimic. Examples of an environmental mimic include O₂ tension, CO₂ tension, hydrostatic pressure, osmotic pressure, pH balance, ultraviolet exposure, temperature exposure or other physico-chemical manipulations.

### Phenotypic Assays

### Assays for Cell Sequencing Data

An example type of phenotypic assay data is cell sequencing data. Examples of cell sequencing data include DNA sequencing data or RNA sequencing data e.g., transcript-level sequencing data. In various embodiments, the cell sequencing data is expressed as a FASTA format file, BAM file, or a BLAST output file. The cell sequencing data obtained from a cell may include one or more differences in comparison to a reference sequence (e.g., a control sequence, a wild-type sequence, or a sequence of healthy individuals). Differences may include variants, mutations, polymorphisms, insertions, deletions, knock-ins, and knock-outs of one or more nucleotide bases. In various embodiments, the differences in the cell sequencing data correspond to high risk alleles that are informative for determining a genetic risk of a disease. In various embodiments, the high risk alleles are highly penetrant alleles.

In various embodiments, the differences between the cell sequencing data and the reference sequence can serve as features for the machine learning model. In various embodiments, one or more sequences of the cell sequencing data, frequency of a nucleotide base or a mutated nucleotide base at a particular position of the cell sequencing data, insertions/deletions/duplications, copy number variations, or a sequence of the sequencing data can serve as features for the machine learning model.

Since many nucleic acids are present in relatively low abundance, nucleic acid amplification greatly enhances the ability to assess expression. The general concept is that nucleic acids can be amplified using paired primers flanking the region of interest. The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form.

Pairs of primers designed to selectively hybridize to nucleic acids corresponding to selected genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals.

Following any amplification, it may be desirable to separate the amplification product from the template and/or the excess primer. Example separation techniques include agarose, agarose-acrylamide or polyacrylamide gel electrophoresis using standard methods (Sambrook et al., 1989). Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Hybridization assays are additionally described in U.S. Pat. No. 5,124,246, which is hereby incorporated by reference in its entirety. In Northern blots, mRNA is separated electrophoretically and contacted with a probe. A probe is detected as hybridizing to an mRNA species of a particular size. The amount of hybridization can be quantitated to determine relative amounts of expression, for example under a particular condition. Probes are used for in situ hybridization to cells to detect expression. Probes can also be used in vivo for diagnostic detection of hybridizing sequences. Probes are typically labeled with a radioactive isotope. Other types of detectable labels can be used such as chromophores, fluorophores, and enzymes. Use of northern blots for determining differential gene expression is further described in U.S. patent application Ser. No. 09/930,213, which is hereby incorporated by reference in its entirety.

### Nucleic Acid Arrays

Microarrays comprise a plurality of polymeric molecules spatially distributed over, and stably associated with, the surface of a substantially planar substrate, e.g., biochips. Microarrays of polynucleotides have been developed and find use in a variety of applications, such as screening, detection of single nucleotide polymorphisms and other mutations, and DNA sequencing. One area in particular in which microarrays find use is in gene expression analysis.

In gene expression analysis with microarrays, an array of “probe” oligonucleotides is contacted with a nucleic acid sample of interest, i.e., target, such as polyA mRNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information. One example of a microarray is a single nucleotide polymorphism (SNP)—Chip array, which is a DNA microarray that enables detection of polymorphisms in DNA.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phosphorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

### Nucleic Acid Sequencing

Various different sequencing methods can be implemented for sequencing nucleic acids (either DNA or RNA). For example, for DNA sequencing any one of whole genome sequencing, whole exome sequencing, or a targeted panel sequencing can be conducted. Whole genome sequencing refers to the sequencing of the entire genome, whole exome sequencing refers to the sequencing of all expressed genes of a genome, and targeted panel sequencing refers to the sequencing of a particular subset of genes in the genome.

For RNA, RNA-seq (RNA Sequencing), also called Whole Transcriptome Shotgun Sequencing (WTSS), is a technology that utilizes the capabilities of next-generation sequencing to reveal a snapshot of RNA presence and quantity from a genome at a given moment in time. An example of a RNA-seq technique is Perturb-seq, which involves a high-throughput method of performing RNA sequencing (e.g., bulk or single-cell RNA sequencing) on pooled perturbation screens. Example Perturb-seq datasets are described in Replogle, J. et al., Mapping information-rich genotype-phenotype landscapes with genome-scale perturb-seq. Cell, 185(14):2559-2575.e28, 2022, which is hereby incorporated by reference in its entirety.

The transcriptome of a cell is dynamic; it continually changes as opposed to a static genome. The recent developments of Next-Generation Sequencing (NGS) allow for increased base coverage of a DNA sequence, as well as higher sample throughput. This facilitates sequencing of the RNA transcripts in a cell, providing the ability to look at alternative gene spliced transcripts, post-transcriptional changes, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries, Ongoing RNA-Seq research includes observing cellular pathway alterations during infection, and gene expression level changes in cancer studies. Prior to NGS, transcriptomics and gene expression studies were previously done with expression microarrays, which contain thousands of DNA sequences that probe for a match in the target sequence, making available a profile of all transcripts being expressed.

### Read Assembly

Two different assembly methods can be used to analyze the raw sequence reads: de-novo and genome-guided.

The first approach does not rely on the presence of a reference genome in order to reconstruct the nucleotide sequence. Due to the small size of the short reads de novo assembly may be difficult though some software does exist (Velvet (algorithm), Oases, and Trinity to mention a few), as there cannot be large overlaps between each read needed to easily reconstruct the original sequences. The deep coverage also makes the computing power to track all the possible alignments prohibitive. This deficit can be improved by using longer sequences obtained from the same sample using other techniques such as Sanger sequencing, and using larger reads as a “skeleton” or a “template” to help assemble reads in difficult regions (e.g., regions with repetitive sequences).

An “easier” and relatively computationally cheaper approach is that of aligning the millions of reads to a “reference genome.” There are many tools available for aligning genomic reads to a reference genome (sequence alignment tools), however, special attention is needed when alignment of a transcriptome to a genome, mainly when dealing with genes having intronic regions. Several software packages exist for short read alignment, and recently specialized algorithms for transcriptome alignment have been developed, e.g. Bowtie for RNA-seq short read alignment, TopHat for aligning reads to a reference genome to discover splice sites, Cufflinks to assemble the transcripts and compare/merge them with others, or FANSe. Additional available algorithms for aligning sequences reads to a reference sequence include basic local alignment search tool (BLAST) and FASTA. These tools can also be combined to form a comprehensive system.

The assembled sequence reads can be used for a variety of purposes including generating a transcriptome and/or identifying mutations, polymorphisms, insertions/deletions, knockins/knockouts and like in the sequence reads.

### Assays for Gene Expression

An example type of phenotypic assay data is gene expression data. In various embodiments, the gene expression data includes quantitative levels of expression for one or more genes, an indication of whether one or more genes are differentially expressed (e.g., higher or lower expression), a ratio of the expression level of a gene in relation to a reference value (e.g., a reference gene expression level in healthy individuals). In various embodiments, these examples of gene expression data can serve as features of the machine learning model. In various embodiments, the expression levels of genes in a previously identified panel of genes can serve as features of the machine learning model. For example, genes in the panel can be previously identified as disease-associated genes when they are differentially expressed.

In various embodiments, the gene expression data can be determined using the cell sequencing data and/or protein expression data. For example, the cell sequencing data may be transcript level sequencing data (e.g., mRNA sequencing data or RNA-seq data). Therefore, the abundance of particular mRNA transcripts can be indicative of the expression level of a corresponding gene that the mRNA transcripts are transcribed from. Differential expression analysis based on mRNA transcription levels can be performed using available tools such as baySeq (Hardcastle, T. et al. baySeq: Empirical Bayesian methods for identifying differential expression in sequence count data. BMC bioinformatics, 11, 1-14 (2010)), DESeq (Anders, S. et al. Differential expression analysis for sequence count data. Genome biology, 11, R106, (2010)), EBSeq (Leng, N. et al. EBSeq: an empirical Bayes hierarchical model for inference in RNA-seq experiments. Bioinformatics, 29, 1035-1043, 2013), edgeR (Robinson, M. D. et al. edgeR: a Bioconductor package for differential expression analysis of digital gene expression data. Bioinformatics, 26, 139-140, (2010)), NBPSeq (Di, Y., et al., The NBP Negative Binomial Model for Assessing Differential Gene Expression from RNA-Seq. Statistical applications in genetics and molecular biology, 10, 1-28 (2011)), SAMseq (Li, J. et al. Finding consistent patterns: a nonparametric approach for identifying differential expression in RNA-Seq data. Statistical methods in medical research, 22, 519-536, (2013)), ShrinkSeq (Van De Wiel, M. A. et al. Bayesian analysis of RNA sequencing data by estimating multiple shrinkage priors. Biostatistics, 14, 113-128 (2013)), TSPM (Auer, P. L. et al. A Two-Stage Poisson Model for Testing RNA-Seq Data. Statistical applications in genetics and molecular biology, 10 (2011), voom (Law, C. W. et al. voom: Precision weights unlock linear model analysis tools for RNA-seq read counts. Genome biology, 15, R29 (2014)), limma (Smyth, G. K. Linear models and empirical bayes methods for assessing differential expression in microarray experiments. Statistical applications in genetics and molecular biology, 3, Article3 (2004)), PoissonSeq (Li, J. et al. Normalization, testing, and false discovery rate estimation for RNA-sequencing data. Biostatistics, 13, 523-538 (2012)), DESeq2 (Love, M. I. et al. Moderated estimation of fold change and dispersion for RNA-seq data with DESeq2. Genome biology, 15, 550 (2014)), and ODP (Storey, J. D. The optimal discovery procedure: a new approach to simultaneous significance testing. Journal of the Royal Statistical Society: Series B (Statistical Methodology), 69, 347-368 (2007)), each of which is hereby incorporated in its entirety.

As another example, the protein expression data may also serve as a readout for levels of gene expression. Expression levels of a protein may correspond to levels of mRNA transcripts from which the protein is translated from. Again, the levels of mRNA transcripts can be indicative of the expression level of a corresponding gene. In some embodiments, both cell sequencing data and protein expression data is used to determine gene expression data, given that there are post-transcriptional modifications and post-translational modifications that can result in differing levels of mRNA and protein.

### Assays for Imaging and Immunohistochemistry

An example type of phenotypic assay data includes microscopy data, such as high-resolution microscopy data and/or immunohistochemistry imaging data. Microscopy data can be captured using a variety of different imaging modalities including confocal microscopy, super-high-resolution microscopy, in vivo two photon microscopy, electron microscopy (e.g., scanning electron microscopy or transmission electron microscopy), atomic force microscopy, bright field microscopy, and phase contrast microscopy. In various embodiments, microscopy data captured from microscopy images can serve as features for the machine learning model.

In various embodiments, the microscopy data represent high dimensional data that, without machine learning implemented analysis, would be difficult to relate to diseased or normal cell phenotypes. Examples of microscopy data can include microscopy images, antibody staining for specific markers, imaging of ions (e.g., sodium, potassium, calcium), division rate of cells, number of cells, environmental surroundings of a cell, and presence or absence of diseased markers (e.g., in immunohistochemistry images, markers of inflammation, degeneration, cellular swelling/shriveling, fibrosis, macrophage recruitment, immune cells). Examples of imaging analysis tools for analyzing microscopy data include CellPAINT (e.g., including cell-specific Paint assays such as NeuroPAINT), pooled optical screening (POSH), and CellProfiler.

As one example, for fluorescent imaging, the cells can be stained using fluorescently tagged antibodies (e.g., primary antibody and secondary antibody with a fluorescent tag). In particular embodiments, the cells can be stained such that different cellular components can be readily distinguished in the subsequently captured images. For example, cellular component specific stains can be used (e.g., DAPI or Hoechst for nuclear stains, Phalloidin for actin cytoskeleton, wheat germ agglutinin (WGA) for Golgi/plasma membrane, MitoFISH for mitochondria, and BODIPY for lipid droplets). In various embodiments, fluorescent dyes may be programmable such that the presence of the fluorescence indicates the presence of a particular phenotype. For example, in vitro cells may be treated with a fluorescent reporter (e.g., green fluorescent protein reporter) such that the presence of the phenotype corresponds to the expression of the fluorescent reporter. Here, a plasmid encoding for the fluorescent reporter may be delivered to the cells to stably transfect the cells and serve as a measure of gene expression. Therefore, observance of the fluorescent reporter protein indicates expression of the gene, which can correspond to a particular phenotype of a disease. For example, overexpression or under expression of a protein product corresponding to the gene can indicate the presence of a disease. In various embodiments, multiple cellular stains can be used together with limited interference across channels, thereby enabling the visualization of several different cellular components in one image. For example, preparation of cells can involve the use of Cell Painting, which is a morphological profiling assay that multiplexes six fluorescent dyes that can be imaged across five channels for identifying eight cellular components. Different versions of Cell Painting can be developed and used depending on the type of cells that are to be imaged. For example, for brain cells, a custom version of CellPaint, hereafter termed NeuroPaint, can be employed to image for various cellular components of brain cells. Images can be captured using any suitable fluorescent imaging including confocal imaging and two-photon microscopy.

In some scenarios, in vitro cells are plated in wells and then stained e.g., using primary/secondary antibodies that are fluorescently tagged. In some embodiments, the in vitro cells are fixed prior to imaging. In some embodiments, the in vitro cells can undergo live cell imaging to observe changes in the cellular phenotypes over time.

For confocal microscopy, tissues or tissue organoids are embedded in optimal tissue cutting compound and frozen at −20° C. Once frozen, tissues are sliced using a microtome (e.g., 5-50 microns in thickness). Tissue slices are mounted on glass slides. Tissue slices are stained and fixed to prepare them for imaging. In some embodiments, tissues are treated using blocking buffer to block for non-specific staining between a primary antibody and the tissue. Example blocking buffer can include 1% horse serum in phosphate buffered saline. Primary antibodies are diluted to appropriate dilutions and applied to the tissue sections. Tissue slices are washed, then incubated with a secondary antibody specific for the primary antibody. In some embodiments, the primary antibody and/or secondary antibody are fluorescently tagged. Tissue slices are washed and prepared for imaging. Tissue slices can then be imaged using fluorescent (e.g., confocal) microscopy.

For immunohistochemistry, tissues are fixed, paraffin embedded, and cut. Generally, tissues are fixed using a formaldehyde fixation solution. Tissues are dehydrated by immersing them consecutively in increasing concentrations of ethanol (e.g., 70%, 90%, 100% ethanol) and then immersed in xylene. Tissues are embedded in paraffin and then cut into tissue sections (e.g., 5-15 microns in thickness). This can be accomplished using a microtome. Tissue sections are mounted onto histological slides, and then dried.

Paraffin embedded sections can then be stained for particular targets (e.g., proteins, biomarkers) of interest. Sections are rehydrated (e.g., in decreasing concentrations of ethanol—100%, 95%, 70%, and 50% ethanol) and then rinsed with deionized H₂O. If needed, tissues are treated using blocking buffer to block for non-specific staining between a primary antibody and the tissue. Example blocking buffer can include 1% horse serum in phosphate buffered saline. Primary antibodies are diluted to appropriate dilutions and applied to the tissue sections. Tissue slices are washed, then incubated with a secondary antibody specific for the primary antibody. Tissue slices are washed, and then mounted. Tissue slices can then be imaged using microscopy (e.g., bright field microscopy, phase contrast microscopy, or fluorescence microscopy). Additional methods for performing immunohistochemistry are described in further detail in Simon et al, BioTechniques, 36(1):98 (2004) and Haedicke et al., BioTechniques, 35(1): 164 (2003), each of which is hereby incorporated by reference in its entirety. In various embodiments, immunohistochemistry can be automated using commercially available instruments, such as the Benchmark ULTRA system available from the Roche Group.

### Assays for In Situ Sequencing

An example type of phenotypic assay data includes images captured from in situ sequencing. Generally, in situ sequencing refers to the sequencing of nucleic acids in the preserved context of fixed cells and/or tissues. Thus, in situ sequencing enables the reading of sequences directly from intact cells and/or tissues, quantifies large numbers of mRNA transcripts simultaneously, and spatially resolves them with single-cell resolution. In situ sequencing can be applied for transcription expression profiling, splice variant mapping, mutation detection, and cellular genotyping (e.g., sequencing of barcode sequences to identify corresponding perturbations).

In various embodiments, cells or tissues are first fixed prior to performing sequencing to retain the spatial context of the cells or tissues. Example fixatives include crosslinkers such as formaldehyde, paraformaldehyde, and glutaraldehyde. Cells or tissues can further undergo a permeabilization step. Example reagents for permeabilization of cells or tissues include ethanol, methanol, acetone, saponin, Triton X-100, and Tween-20.

In various embodiments, in situ sequencing is fluorescent in situ sequencing (FISSEQ). FISSEQ combines the spatial context of RNA-FISH and the global transcriptome profiling of RNA-seq. FISSEQ involves preserving the cell and/or tissue, thereby enabling single molecule in situ RNA localization. Generally, FISSEQ involves a series of wet lab processing steps e.g., single-base polymerase extensions, which are performed on fixed cells or tissues. FISSEQ is analogous to sequencing by synthesis methods, except that FISSEQ is performed in situ (e.g., on fixed cells or tissues). Sequencing by synthesis is further described in U.S. Pat. Nos. 5,302,509 and 10,793,904, each of which is incorporated by reference in its entirety. In various embodiments, any sequencing methodology which relies on successive incorporation of nucleotides into a polynucleotide chain can be used. Suitable techniques in addition to FISSEQ include, for example, Pyrosequencing, MPSS (massively parallel signature sequencing) sequencing by synthesis, sequencing by ligation, sequencing by hybridization, and sequencing by cyclic reversible polymerization hybridization chain reaction (HCR).

In various embodiments, in situ sequencing involves the use of modified nucleotides that act as chain terminators. These modified nucleotides are also referred to as tagged, reversibly terminated bases. Once the modified nucleotide has been incorporated into the growing polynucleotide chain complementary to an amplicon sequence being sequenced, there is no free 3′-OH group available to direct further sequence extension. Once the nature of the base incorporated into the growing chain has been determined, the 3′ block may be removed to allow addition of the next successive nucleotide. By ordering the products derived using these modified nucleotides, the sequence of the amplicon can be determined. In various embodiments, each of the modified nucleotides is labeled using a different label, known to correspond to the particular base, to facilitate discrimination between the bases added at each incorporation step. In various embodiments, modified nucleotides are labeled using fluorescent labels. Each nucleotide base type (e.g., adenine, thymine, guanine, cytosine) may carry a different fluorescent label. In some embodiments, the detectable label need not be a fluorescent label and any label which allows the detection of the incorporation of the nucleotide can be used.

In various embodiments, labels of the incorporated modified nucleotides are detected by using laser light of a wavelength specific for the labeled nucleotides, or the use of other suitable sources of illumination. For example, the fluorescence from the label on the nucleotide may be detected by a camera or other suitable detection means. In various embodiments, an entire sample can be imaged at each cycle to identify the fluorescent label, thereby identifying the incorporated nucleotide base. The fluorescent labels are then cleaved and washed away (e.g., via a stripping reagent which cleaves off base terminators and fluorophores), and the next cycle is initiated. The nucleotide sequence of each amplicon is thus read out in-situ via fluorescent microscopy. Further description of FISSEQ is detailed in Lee et al. “Fluorescent in situ sequencing (FISSEQ) of RNA for gene expression profiling in intact cells and tissues,” Nature Protocols, 10, 442-458 (2015), which is incorporated by reference in its entirety.

### Systems and Computing Devices

In various embodiments, the methods described herein, including the methods of training and deploying machine learning models for generating predictions of cellular responses to perturbations are performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

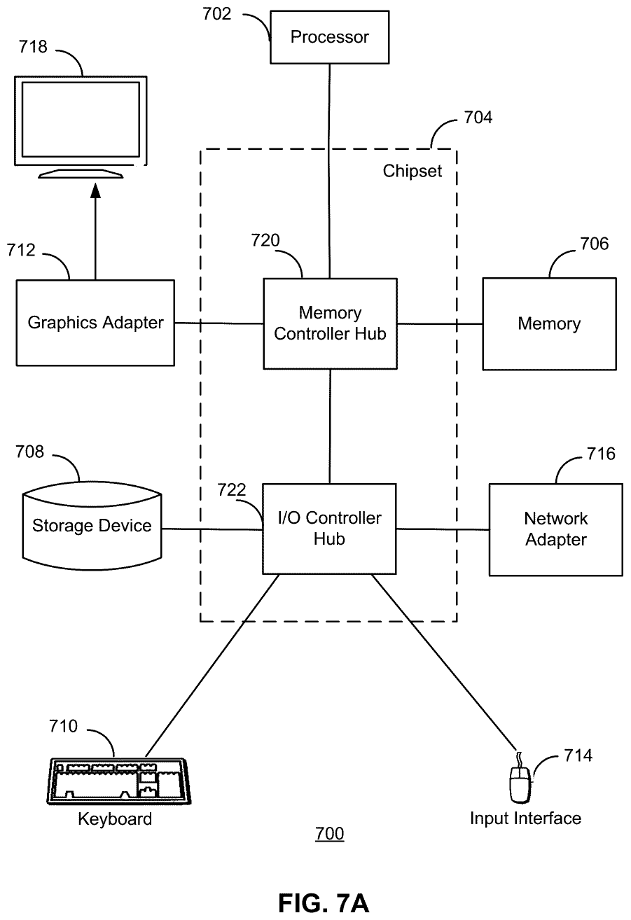

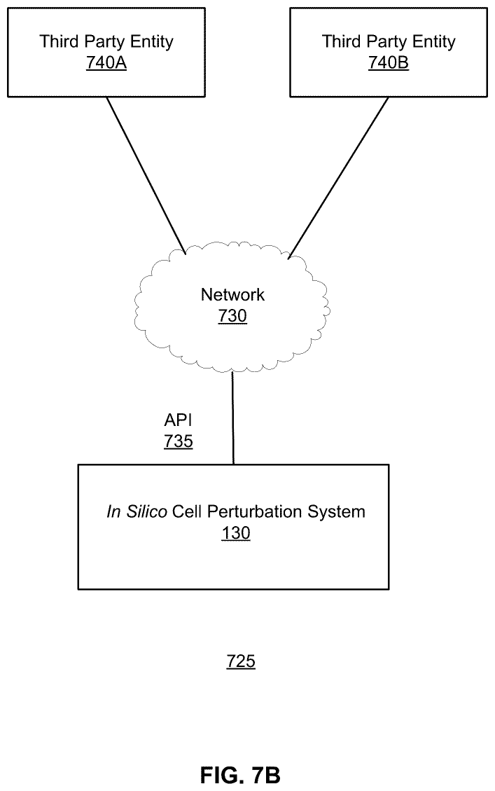

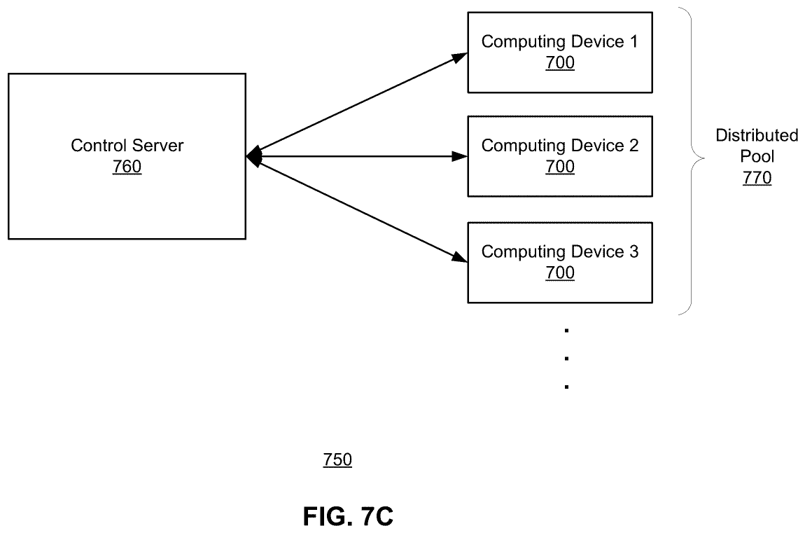

FIG. 7A illustrates an example computing device for implementing the system and methods described in FIGS. 1-3, 4A-4D, 5, and 6A. Furthermore, FIG. 7B depicts an overall system environment for implementing an in silico cell perturbation system, in accordance with an embodiment. FIG. 7C is an example depiction of a distributed computing system environment for implementing the system environment of FIG. 7B.

In some embodiments, the computing device 700 shown in FIG. 7A includes at least one processor 702 coupled to a chipset 704. The chipset 704 includes a memory controller hub 720 and an input/output (I/O) controller hub 722. A memory 706 and a graphics adapter 712 are coupled to the memory controller hub 720, and a display 718 is coupled to the graphics adapter 712. A storage device 708, an input interface 714, and network adapter 716 are coupled to the I/O controller hub 722. Other embodiments of the computing device 700 have different architectures.

The storage device 708 is a non-transitory computer-readable storage medium such as a hard drive, compact disk read-only memory (CD-ROM), DVD, or a solid-state memory device. The memory 706 holds instructions and data used by the processor 702. The input interface 714 is a touch-screen interface, a mouse, track ball, or other type of input interface, a keyboard, or some combination thereof, and is used to input data into the computing device 700. In some embodiments, the computing device 700 may be configured to receive input (e.g., commands) from the input interface 714 via gestures from the user. The graphics adapter 712 displays images and other information on the display 718. The network adapter 716 couples the computing device 700 to one or more computer networks.

The computing device 700 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 708, loaded into the memory 706, and executed by the processor 702.

The types of computing devices 700 can vary from the embodiments described herein. For example, the computing device 700 can lack some of the components described above, such as graphics adapters 712, input interface 714, and displays 718. In some embodiments, a computing device 700 can include a processor 702 for executing instructions stored on a memory 706.

In various embodiments, the different entities depicted in FIG. 7B may implement one or more computing devices to perform the methods described above, including the methods of training and deploying one or more machine learning models. For example, the in silico cell perturbation system 130, third party entity 740A, and third party entity 740B may each employ one or more computing devices. As another example, one or more of the sub-systems of the in silico cell perturbation system 130 (as shown in FIG. 1B) may employ one or more computing devices to perform the methods described above.

The methods of training and deploying one or more machine learning models can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of a machine learning model disclosed herein.

Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that is capable of recording and reproducing the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### System Environment

FIG. 7B depicts an overall system environment for implementing a compound-target analysis system, in accordance with an embodiment. The overall system environment 725 includes an in silico cell perturbation system 130, as described earlier in reference to FIG. 1, and one or more third party entities 740A and 740B in communication with one another through a network 730. FIG. 7A depicts one embodiment of the overall system environment 700. In other embodiments, additional or fewer third party entities 740 in communication with the in silico cell perturbation system 130 can be included. Generally, the in silico cell perturbation system 130 trains or deploys machine learning models that make predictions, e.g., predictions of cellular responses to perturbations. The third party entities 740 communicate with the in silico cell perturbation system 130 for purposes associated with implementing the machine learning models or obtaining predictions or results from the machine learning models.

In various embodiments, the methods described above as being performed by the in silico cell perturbation system 130 can be dispersed between the in silico cell perturbation system 130 and third party entities 740. For example, a third party entity 740A or 740B can generate phenotypic assay data by performing one or more phenotypic assays. Referring again to FIG. 1, a third party entity 740A or 740B can provide a perturbation 115 to cell 110A to induce a diseased state in a perturbed cell 110B. Additionally, the same or different third party entity 740A or 740B can capture phenotypic assay data from the cell 110A and/or perturbed cell 110B and provide the phenotypic assay data to the in silico cell perturbation system 130 for training machine learning models.

In various embodiments, the in silico perturbation system 130 can be operated by a single entity. For example, the single entity may perform the training and deployment of machine learning models to generate in silico predictions of cellular responses to perturbations. In other embodiments, the third party entity third party entity 740A or 740B additionally perform the training of machine learning models using the phenotypic assay data. The third party entity 740A or 740B provides the trained machine learning models to another entity to deploy the machine learning models to generate predictions of cellular responses to perturbations.

### Third Party Entity

In various embodiments, the third party entity 740 represents a partner entity of the in silico cell perturbation system 130 that operates either upstream or downstream of the in silico cell perturbation system 130. As one example, the third party entity 740 operates upstream of the in silico cell perturbation system 130 and provide information to the in silico cell perturbation system 130 to enable the training of machine learning models. In this scenario, the in silico cell perturbation system 130 receives data, such as phenotypic assay data collected by the third party entity 740. For example, the third party entity 740 may have performed the methodologies shown in FIG. 1 (e.g., exposed cell 110A to a perturbation 115 and performed a phenotype assay 120 to capture phenotypic assay data from the cell 110A and/or perturbed cell 110B). The third party entity 740 may provide the phenotypic assay data to the in silico cell perturbation system 130.

As another example, the third party entity 740 operates downstream of the in silico cell perturbation system 130. In this scenario, the in silico cell perturbation system 130 may generate predictions of cellular responses to perturbations and provides information relating to the predicted cellular responses to the third party entity 740. The third party entity 740 can subsequently use the information.

### Network

This disclosure contemplates any suitable network 730 that enables connection between the in silico cell perturbation system 130 and third party entities 740. The network 730 may comprise any combination of local area and/or wide area networks, using both wired and/or wireless communication systems. In one embodiment, the network 730 uses standard communications technologies and/or protocols. For example, the network 730 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 730 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 730 may be represented using any suitable format, such as hypertext markup language (HTML) or extensible markup language (XML). In some embodiments, all or some of the communication links of the network 730 may be encrypted using any suitable technique or techniques.

### Application Programming Interface (API)

In various embodiments, the in silico cell perturbation system 130 communicates with third party entities 740A or 740B through one or more application programming interfaces (API) 735. The API 735 may define the data fields, calling protocols and functionality exchanges between computing systems maintained by third party entities 740 and the in silico cell perturbation system 130. The API 735 may be implemented to define or control the parameters for data to be received or provided by a third party entity 740 and data to be received or provided by the in silico cell perturbation system 130. For instance, the API may be implemented to provide access only to information generated by one of the subsystems comprising the in silico cell perturbation system 130. The API 735 may support implementation of licensing restrictions and tracking mechanisms for information provided by in silico cell perturbation system 130 to a third party entity 740. Such licensing restrictions and tracking mechanisms supported by API 735 may be implemented using blockchain-based networks, secure ledgers and information management keys. Examples of APIs include remote APIs, web APIs, operating system APIs, or software application APIs.

An API may be provided in the form of a library that includes specifications for routines, data structures, object classes, and variables. In other cases, an API may be provided as a specification of remote calls exposed to the API consumers. An API specification may take many forms, including an international standard such as POSIX, vendor documentation such as the Microsoft Windows API, or the libraries of a programming language, e.g., Standard Template Library in C++ or Java API. In various embodiments, the in silico cell perturbation system 130 includes a set of custom API that is developed specifically for the in silico cell perturbation system 130 or the subsystems of the in silico cell perturbation system 130.

### Distributed Computing Environment

In some embodiments, the methods described above, including the methods of training and implementing one or more machine learning models, are, performed in distributed computing system environments where local and remote computer systems, which are linked (either by hardwired data links, wireless data links, or by a combination of hardwired and wireless data links) through a network, both perform tasks. In some embodiments, one or more processors for implementing the methods described above may be located in a single geographic location (e.g., within a home environment, an office environment, or a server farm). In various embodiments, one or more processors for implementing the methods described above may be distributed across a number of geographic locations. In a distributed computing system environment, program modules may be located in both local and remote memory storage devices.

FIG. 7C is an example depiction of a distributed computing system environment for implementing the system environment of FIG. 7B. The distributed computing system environment 750 can include a control server 760 connected via communications network with at least one distributed pool 770 of computing resources, such as computing devices 700, examples of which are described above in reference to FIG. 7A. In various embodiments, additional distributed pools 770 may exist in conjunction with the control server 760 within the distributed computing system environment 750. Computing resources can be dedicated for the exclusive use in the distributed pool 770 or shared with other pools within the distributed processing system and with other applications outside of the distributed processing system. Furthermore, the computing resources in distributed pool 770 can be allocated dynamically, with computing devices 700 added or removed from the pool 710 as necessary.

In various embodiments, the control server 760 is a software application that provides the control and monitoring of the computing devices 700 in the distributed pool 770. The control server 760 itself may be implemented on a computing device (e.g., computing device 700 described above in reference to FIG. 7A). Communications between the control server 760 and computing devices 700 in the distributed pool 770 can be facilitated through an application programming interface (API), such as a Web services API. In some embodiments, the control server 760 provides users with administration and computing resource management functions for controlling the distributed pool 770 (e.g., defining resource availability, submission, monitoring and control of tasks to performed by the computing devices 700, control timing of tasks to be completed, ranking task priorities, or storage/transmission of data resulting from completed tasks).

In various embodiments, the control server 760 identifies a computing task to be executed across the distributed computing system environment 750. The computing task can be divided into multiple work units that can be executed by the different computing devices 700 in the distributed pool 770. By dividing up and executing the computing task across the computing devices 700, the computing task can be effectively executed in parallel. This enables the completion of the task with increased performance (e.g., faster, less consumption of resources) in comparison to a non-distributed computing system environment.

In various embodiments, the computing devices 700 in the distributed pool 770 can be differently configured in order to ensure effective performance for their respective jobs. For example, a first set of computing devices 700 may be dedicated to performing collection and/or analysis of phenotypic assay data. A second set of computing devices 700 may be dedicated to performing the training of machine learning models. The first set of computing devices 700 may have less random access memory (RAM) and/or processors than the second set of second computing devices 700 given the likely need for more resources when training the machine learning models.

The computing devices 700 in the distributed pool 770 can perform, in parallel, each of their jobs and when completed, can store the results in a persistent storage and/or transmit the results back to the control server 760. The control server 760 can compile the results or, if needed, redistribute the results to the respective computing devices 700 to for continued processing.

In some embodiments, the distributed computing system environment 750 is implemented in a cloud computing environment. In this description, “cloud computing” is defined as a model for enabling on-demand network access to a shared set of configurable computing resources. For example, the control server 760 and the computing devices 700 of the distributed pool 770 may communicate through the cloud. Thus, in some embodiments, the control server 760 and computing devices 700 are located in geographically different locations. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### EXAMPLES

### Example 1: Developing Generative Model for Modeling Perturbations as Sparse Latent Offsets that Compose Additively

### Example Methods

The goal was build a generative model for perturbation effects, hereafter referred to as “Sparse Additive Mechanism Shift Variational Autoencoder” or “SAMS-VAE.” SAMS-VAE models perturbations as inducing sparse latent offsets that compose additively. Datasets (xᵢ, dᵢ)ᵢ₌₁^{N} for observations xᵢ∈{0,1}^{T}, where d_{i,j} is 1 if sample i received perturbation j and 0 otherwise. This example builds generative models, such as SAMS-VAE, p(xᵢ|dᵢ).

Generative models have the following basic structure:

$${z}_{i} = {z}_{i}^{b} + {z}_{i}^{p}$$

$${x}_{i} ∼ p ( {x}_{i} \vert {z}_{i} ; θ )$$

zᵢ∈R^{D}^{_{z}} is the latent state embedding for sample i, which was modeled as the sum of a latent basal state zᵢ^{b}∈R^{D}^{_{z}} and a latent perturbation effect embedding zᵢ^{p} ∈R^{D}^{_{z}}. Observations were then sampled from a conditional likelihood p(xᵢ|zᵢ; θ). Parametric likelihoods were implemented for p(xᵢ|zᵢ; θ), where parameters were computed from zᵢ using a neural network with parameters θ.

The core modeling assumption of SAMS relates to the distribution p(zᵢ^{p}|dᵢ): perturbations were modeled as inducing sparse latent offsets that compose additively (sparse additive mechanism shift). Specifically, the modeling is as follows:

$${z}_{i}^{p} = {Σ}_{t = 1}^{T} {d}_{i , t} ( {e}_{t} ⊙ {m}_{t} ) \qquad ( 1 )$$

eₜ∈R^{D}^{_{z}} and mₜ∈{0,1}^{D}^{_{z}} are latent variables that determine the latent offset due to perturbation t. mₜ is a binary mask that performs feature selection on the latent offset eₜ; when mₜ is sparse, eₜ ⊙mₜ will result in a sparse offset. eₜ and mₜ are shared across all cells that receive perturbation t.

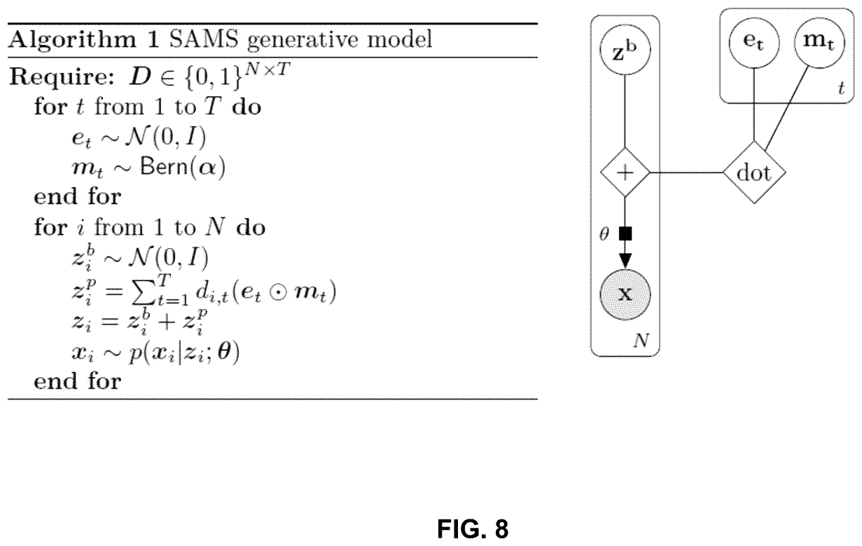

The full generative model is defined as shown in FIG. 8. Specifically, FIG. 8 shows the SAMS generative model represented as an algorithm (left panel) and as a bayesian network and directed factor graph (right panel). The joint probability distribution over the observed and latent variables is defined as:

$$p ( X , {Z}^{b} , M , E \vert D ; θ ) = [ {\prod }_{t = 1}^{T} p ( {e}_{t} ) ( p ( {m}_{t} ) ] [ {\prod }_{i = 1}^{N} p ( {z}_{i}^{b} ) p ( {x}_{i} \vert {z}_{i}^{b} , {d}_{i} , M , E ; θ ) ] \qquad ( 2 )$$

for observations X∈R^{N×D}^{ₓ}, perturbation dosages D∈{0,1}^{N×D}^{ₓ}, latent basal states Z^{b} ∈R^{N×D}^{_{z}}, latent perturbation embeddings E∈R^{T×D}^{_{z}}, and latent perturbation masks M∈{0,1}^{N×D}^{_{z}}.

To build the model class SAMS-VAE, inference was performed on the SAMS generative model using stochastic variation inference. Explicitly, training consists of maximizing the evidence lower bound (ELBO) with respect to model parameters θ and variational distribution parameters ϕ using stochastic gradient descent. θ contains the model decoder parameters and ϕ consists of inferred variational parameters for the mask, embedding, and basal state encoder (described in more detail below). The evidence lower bound (ELBO) for SAMS-VAE is defined as follows:

$$E L B O ( ϕ ) = {E}_{{Z}^{b} , E , M \sim q ( . \vert X , D ; ϕ )} log \frac{p ( X , {Z}^{b} , M , E \vert D ; θ )}{q ( {Z}^{b} , M , E \vert X , D ; ϕ )} \qquad ( 3 )$$

During training, observed measurements X (e.g., RNA-seq or imaging data) and annotated perturbation dosages D were provided as input, an estimate for the ELBO was computed using the generative model and variational distribution, and a gradient step with respect to θ and ϕ were taken to maximize the ELBO. Multiple parameterizations of the variational distribution q were computed. As a baseline, the following mean-field inference scheme for SAMS-VAE was considered:

$$q ( {Z}^{b} , M , E \vert X , D ; ϕ ) = [ {\prod }_{t = 1}^{T} q ( {m}_{t} ; ϕ ) q ( {e}_{t} ; ϕ ) ] [ {\prod }_{i = 1}^{N} q ( {z}_{i}^{b} \vert {x}_{i} ; ϕ ) \qquad ( 4 )$$

q(mₜ; ϕ)=Bern(\hat{p}) and q(eₜ; ϕ)=N(ûₜ,\hat{σ}ₜ) were parameterized with learnable parameters \hat{p}ₜ, \hat{μ}ₜ, \hat{σ}ₜ. Gradients were computed for q(mₜ; ϕ) with a Gumbel-Softmax straight-through estimator and q(zᵢ^{b}|xᵢ)=N(\hat{f}_{enc}(xᵢ)) for learnable neural network encoder \hat{f}_{enc} that predicts mean and standard deviation parameters.

Two additional improvements were implemented to the mean-field inference scheme that aim to more faithfully invert the generative model. First, correlations between z^{b} and the latent masks and embeddings were modeled (correlated encoder). This involved implementing q(zᵢ^{b}|xᵢ, dᵢ, E, M)=N(\hat{f}_{enc}([xᵢzᵢ^{p}])) for zᵢ^{p} as defined in Equation (1), where \hat{f}_{enc} is a neural network that takes as input both the observations and the estimated latent perturbation embedding for a given sample. Second, possible correlations between the mask and embedding were modeled by replacing q(eₜ) with q(eₜ|mₜ) (correlated embeddings). This involved implementing q(eₜ|mₜ)=N(\hat{f}_{emb}(mₜ, t)) with learnable neural network \hat{f}_{emb} that predicts the embedding from a mask and a one hot encoding of the treatment index. Applying both of these modifications, the correlated inference scheme for SAMS-VAE is defined as:

$$q ( {Z}^{b} , M , E \vert X , D ) = [ {\prod }_{t = 1}^{T} q ( {m}_{t} ) q ( {e}_{t} \vert {m}_{t} ) ] [ {\prod }_{i = 1}^{N} q ( {z}_{i}^{b} \vert {x}_{i} , {d}_{i} , E , M ) \qquad ( 5 )$$

### Additional Example Models

The performance of the SAMS-VAE generative model was evaluated against additional example models. In particular, additional example models include 1) a compositional perturbation autoencoder variational autoencoder (CPA-VAE) model, and 2) SVAE+.

Referring first to CPA, the CPA method is disclosed in Lotfollahi, M. et al., Learning interpretable cellular responses to complex perturbations in high-throughput screens. bioRxiv, 2021.04.14.439903, which is hereby incorporated by reference in its entirety. Notably, the CPA is not a generative model. Thus, CPA does not specify a prior for the latent basal state, so any predictions must start from an observed cell which is encoded; by contrast, SAMS-VAE specifies prior probability distributions for all variables. This has a few benefits, including 1) allowing for estimates of the data likelihood and 2) allowing simulated samples of observations under perturbations without conditioning on observed cells as reference (we can sample from our prior on latent basal states, while CPA needs to encode other observed cells to generate samples). Third, CPA requires an adversarial network to try to learn latent variables that are not correlated with perturbations (we apply variational inference to our generative model, which encourages this property). As a comparison between SAMS-VAE and the previously published CPA method may not be a representative comparison, an additional model of CPA-VAE was generated. Here, CPA-VAE can be thought of as an extension of CPA to a fully specified generative model. In particular, CPA-VAE is an ablated model with all mask components fixed to 1.

SVAE+ refers to a generative model described in Lopez et al., Learning causal representations of single cells via sparse mechanism shift modeling, 2022, arXiv:2211.03553, which is hereby incorporated by reference in its entirety. SVAE+ is a generative model for modeling perturbation effects in cells, but does not have a mechanism to compose interventions. In contrast, SAMS-VAE models a latent space where perturbations compose additively.

### Quantitative Evaluation of Perturbation Models—Marginal Likelihood

To evaluate the models, consider the marginal log likelihood of held out data under an inferred generative model, estimated via the importance weighted ELBO (IWELBO) as the primary evaluation metric. Specifically, P(X|D; θ, ϕ) was estimated on held out data. Let H=(Z^{b}, M, E) represent the set of latent variables for SAMS-VAE. Then, the importance weighted ELBO with particles is written as:

$$I W E L B O ( X \vert D ; θ , ϕ ) = {E}_{{H}^{1} … , {H}^{k} \sim q ( H \vert X , D ; ϕ )} \frac{p ( X , H \vert D ; θ )}{q ( H \vert X , D ; θ )}$$

The importance weighted ELBO can be used to holistically compare the generalization of generative models such as SAMS-VAE, CPA-VAE, and SVAE+. However, a marginal likelihood cannot be computed for some baseline, such as CPA, which are not fully specific generative models.

### Quantitative Evaluation of Perturbation Models—Average Treatment Effects

Predictive distribution checks were considered as a second category of metrics. Statistics of interest of the predictive distribution of learned models were queried and compared against estimates from the data. These types of assessments can be useful when assessing models for specific use cases, such as predicting the mean of a measurement under different perturbations. Additionally, these metrics can be computed for all models, as they just require samples from the predictive distribution. However, these assessments only characterize narrow aspects of the predictive distribution, providing a less complete assessment than the marginal likelihood. To evaluate perturbation models, focus on the population average treatment effect of a perturbation d* relative to a control perturbation d₀ on each measurement xⱼ:

$$E [ {x}_{j} \vert do ( {d}^{*} ) ] - E [ {x}_{j} \vert do ( {d}_{0} ) ]$$

For example, one may be interested in the average effect of receiving a drug vs placebo on a particular biomarker. Each of these expectations may be estimated from randomized experiments. To create a metric to compare models, compute the coefficient of determination R² between average treatment effect estimates from data and samples from each model's predictive distribution.

### Results

### Application to Perturb-Seq Data

Perturb-seq is a type of biological assay in which cells are exposed to perturbations (e.g., genetic knockouts, where the expression of a gene is disabled) and individually profiled using single cell RNA sequencing (scRNA-seq), which counts gene messenger RNA (mRNA) transcripts in cells. Concretely, the perturb-seq datasets are represented as a gene expression matrix X∈N^{N×D}^{ₓ} and a perturbation dosage matrix T E {0,1}^{N×T} for N cells, D_{z} gene transcripts, and T perturbations. Entries X_{i,j} represent the number of transcripts from gene j observed in cell i and entries T_{i,k} represent whether cell i received perturbation k.

In each experiment, performance of SAMS-VAE was compared with baseline models using shared encoder and decoder architectures, likelihood structures, and hyperparameters.

### Individual Perturbations in a CRISPRi Perturb-seq Screen

A comparison and ablation of each model were performed using two CRISPR interference (CRISPRi) perturb-seq datasets from Replogle, J. et al., Mapping information-rich genotype-phenotype landscapes with genome-scale perturb-seq. Cell, 185(14):2559-2575.e28, 2022, which is hereby incorporated by reference in its entirety. The first, replogle-filtered, analyzed a subset of the genome-wide screen in K562 cells with the addition of cells with non-targeting guides to assess average treatment effects. Specifically, the dataset was filtered to contain cells that received perturbations that were identified as having strong effects and yielding phenotypic clusters. Similarly, the gene expression matrix was filtered to only contain genes that were associated with these strong perturbations. replogle-filtered contains 722 unique guides (perturbations), 118,641 cells, and 1,187 gene expression features per cell. The second dataset, replogle-essential, consists of the data from “essential gene” screen in K562 cells without any additional filtering. This dataset contains 2,167 unique guides, 310,385 cells, and 8,563 gene expression features. Randomly sampled train, validation, and test splits were defined for each dataset (64% train, 16% validation, 20% test). Models were trained with 100 latent dimensions for 1000 epochs with replogle-filtered and 400 epochs with replogle-essential.

Quantitative results of the models are presented in Table 1. SAMS-VAE and CPA-VAE with correlated guides performed the best on the quantitative evaluation across the two datasets, with SAMS-VAE performing the best on replogle-filtered and both achieving evaluation metrics within one standard deviation of the other on replogle-essential. Correlated inference improves the performance of both models across datasets and metrics.

TABLE 1
Quantitative evaluation of models with Replogle datasets

| | | replogle-filtered | | replogle-essential | |
|---|---|---|---|---|---|
| | | | ATE | | ATE |
| Model | Guide | Test IWELBO | r² | Test IWELBO | r² |
| Conditional VAE | Mean Field | −1765.49 ± 0.27 | 0.65 ± 0.02 | −8651.81 ± 0.88 | 0.41 ± 0.00 |
| SVAE+ | Mean Field | −1760.45 ± 0.11 | 0.49 ± 0.01 | −8634.56 ± 0.31 | 0.53 ± 0.03 |
| CPA-VAE | Mean Field | −1758.56 ± 0.24 | 0.19 ± 0.003 | −8631.47 ± 0.76 | 0.15 ± 0.02 |
| SAMS-VAE | Mean Field | −1758.18 ± 0.15 | 0.11 ± 0.002 | −8632.43 ± 0.34 | 0.06 ± 0.00 |
| CPA-VAE | Correlated | −1756.33 ± 0.13 | 0.66 ± 0.01 | −8627.97 ± 0.30 | 0.67 ± 0.01 |
| SAMS-VAE | Correlated | −1755.98 ± 0.06 | 0.72 ± 0.03 | −8628.26 ± 0.58 | 0.67 ± 0.03 |

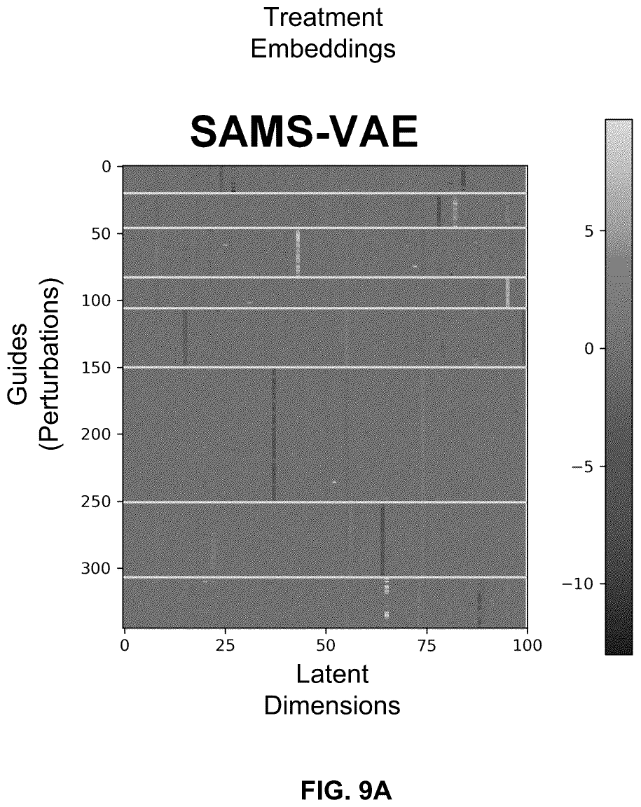

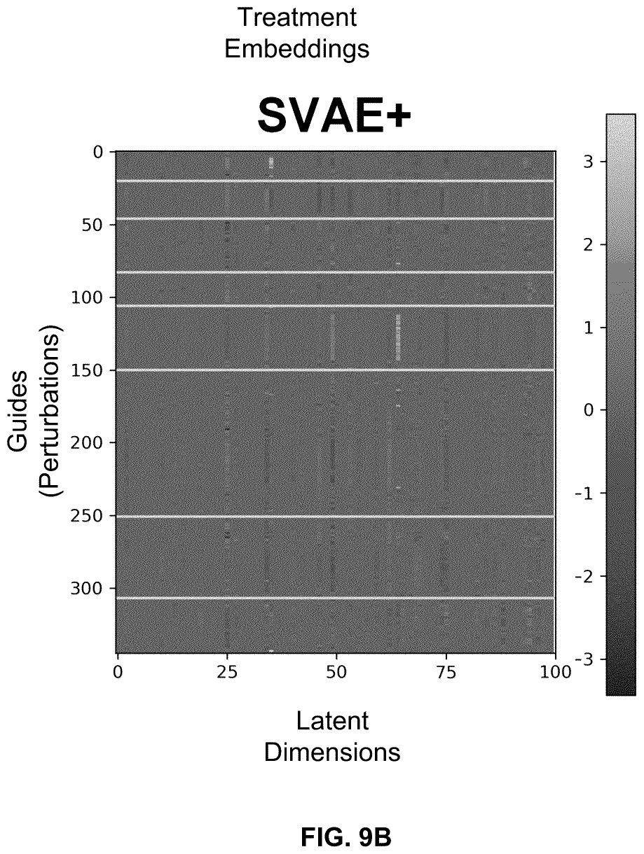

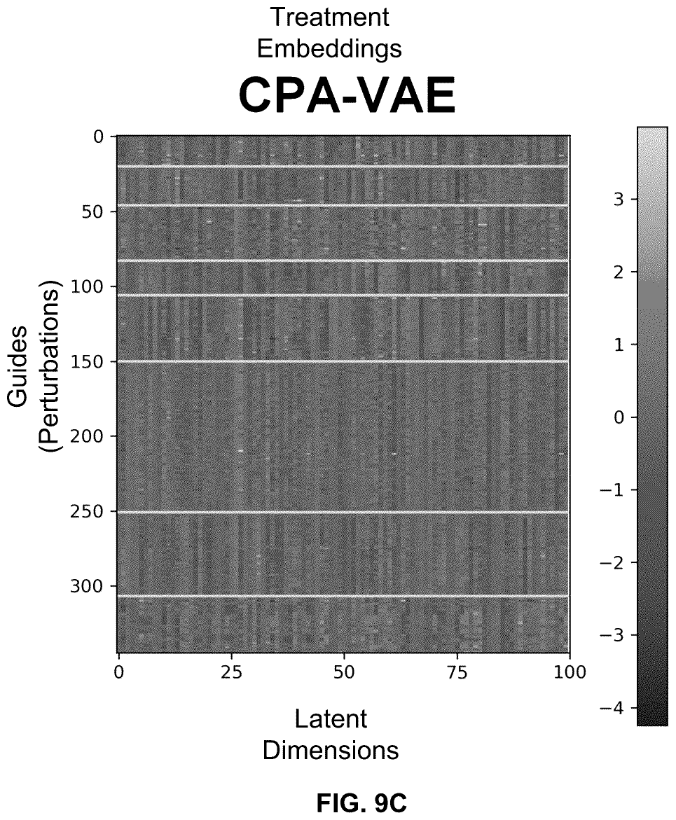

Each model was further characterized by qualitatively comparing the learned latent structures. In Replogle et al. (2022), the authors generated biological pathway annotations for a subset of guides in ‘replogle-filtered’ based on an unsupervised clustering of the data and prior knowledge. Here, the learned treatment embeddings and mask probabilities were visualized for these guides using the SAMS-VAE, SVAE+, and CPA-VAE models with the best test IWELBO results. Specifically, FIGS. 9A-9C show example treatment embeddings for the SAMS-VAE, SVAE+, and CPA-VAE methodologies. FIG. 10 depicts an example learned treatment mask for the SAMS-VAE methodology.

In particular, as shown in FIG. 10, SAMS-VAE learns a sparse latent mask with activation patterns that align with the provided pathway annotations in this dataset. In contrast, CPA-VAE learns a dense embedding space (shown in FIG. 9C), and SVAE+ learns a sparse mask that does not clearly correspond to pathway annotations.

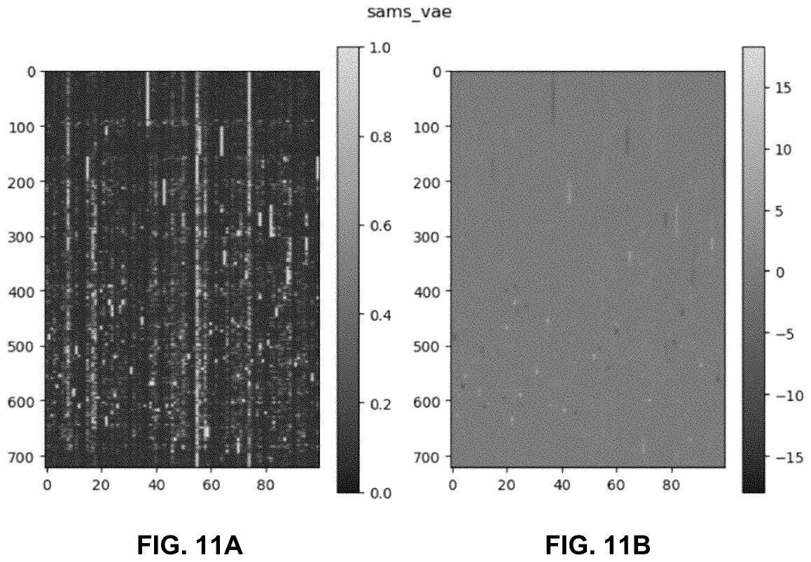

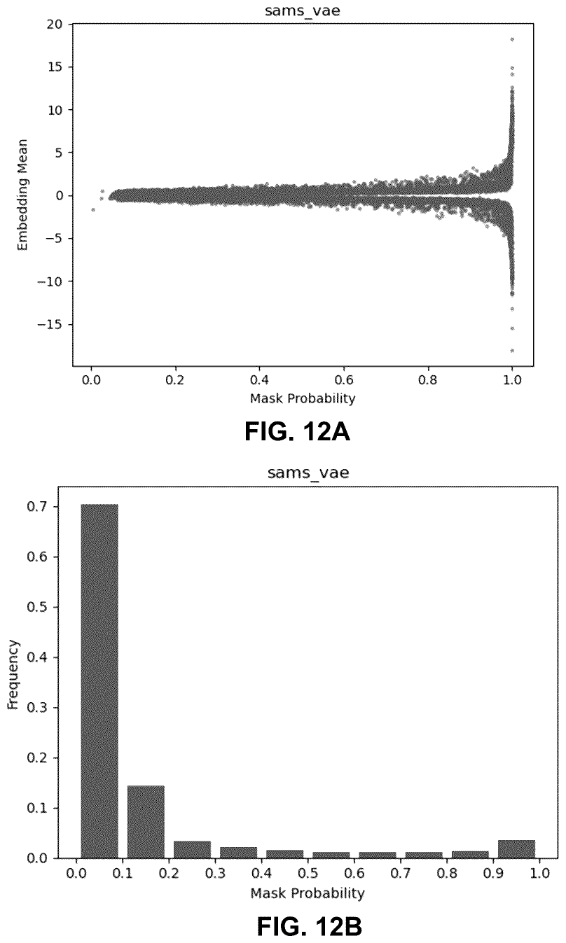

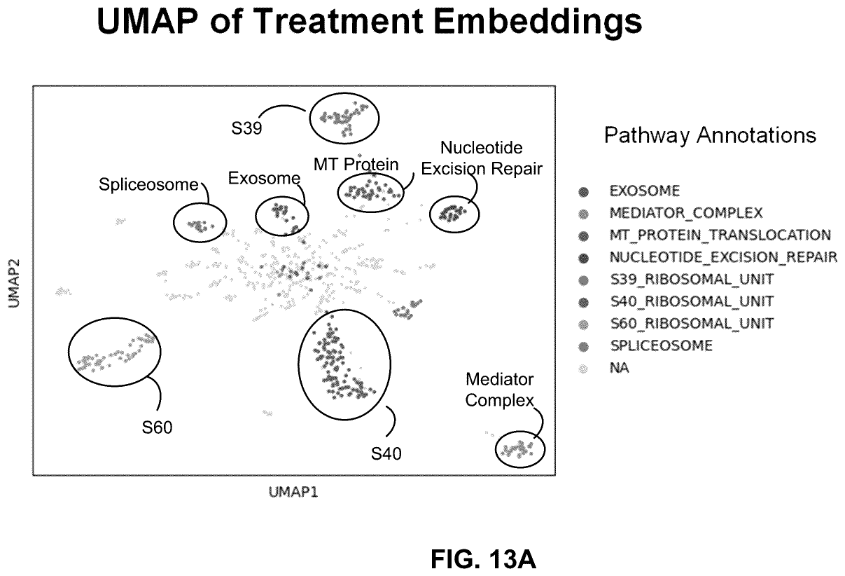

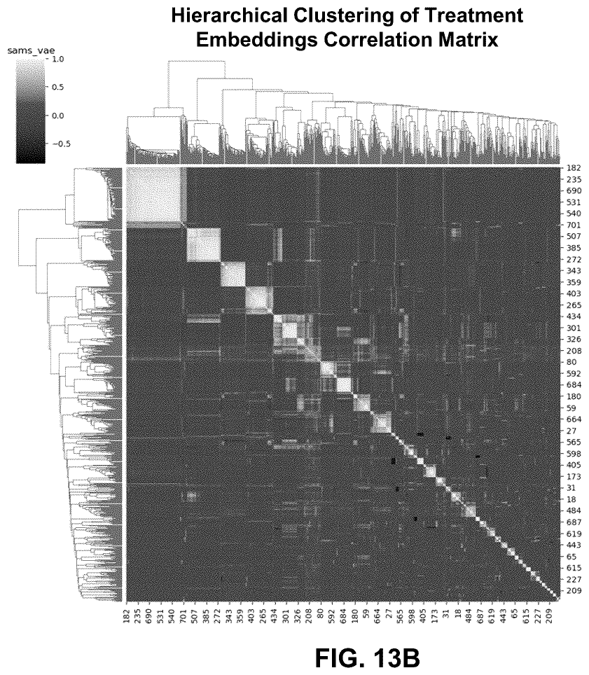

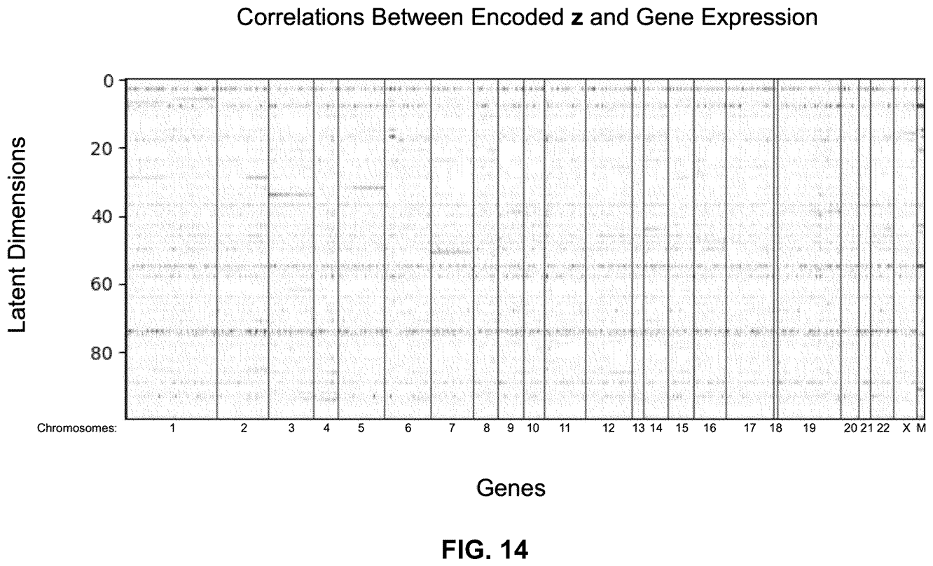

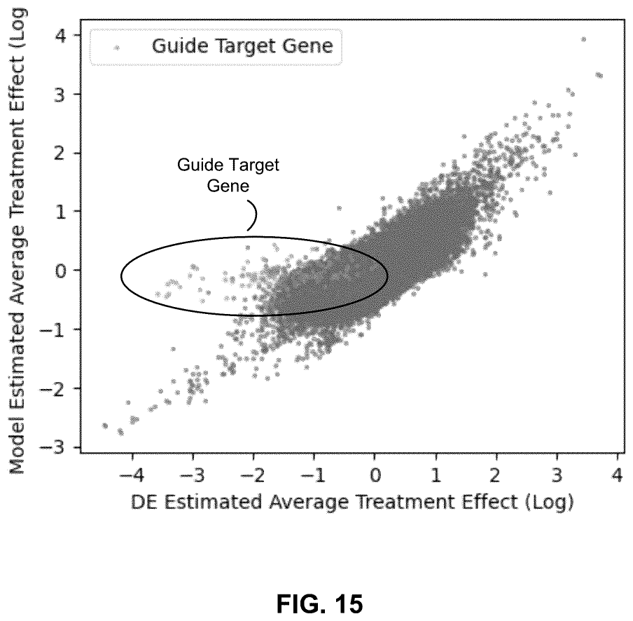

Further analyses of the SAMS-VAE model of replogle-filtered is presented in FIGS. 11A-11B, 12A-12B, 13A-13B, 14, and 15. FIGS. 11A-11B and 12A-12B depict example visualizations of latent mask and embeddings across perturbations, sorted by hierarchical clustering. FIG. 13A shows example UMAP clusters of SAMS-VAE latent treatment embeddings and overlapping pathway annotations, and FIG. 13B shows a hierarchical clustering of the learned latent embeddings. These clusters were strongly enriched for known biological pathways. FIG. 14 shows a heat map of correlations between latent dimensions and gene expression values computed from the encoded dataset. These encoded latent component may be thought of as inferred biomarkers for downstream analyses. Finally, FIG. 15 is an example scatter plot of average treatment effects of each CRISPR guide on the expression of each gene. FIG. 15 visualizes the correlation between data and model estimated average treatment effects. The model-predicted average treatment effect between each perturbation and gene is highly correlated with the observed average treatment effect in the dataset An R² of 0.72 was observed for differentially expressed genes.

### Compositional Interventions in a CRISPRa Perturb-seq Screen

Additionally, a CRISPR activation (CRISPRa) perturb-seq screen was used to assess how effectively SAMS-VAE and each baseline model can learn and predict the effect of perturbation combinations. Further description of the CRISPRa perturb-seq screen is described in Norman, T., et al., Exploring genetic interaction manifolds constructed from rich single-cell phenotypes. Science, 365(6455):786-793, 2019, which is hereby incorporated by reference in its entirety. This screen was specifically designed to explore perturbations that have non-additive effects in combination, making this a challenging setting for modeling combinations. The Norman dataset contains 105 distinct targeting guides applied both on their own and in 131 combinations. In total, the dataset contains 111,255 cells, each with 19,018 measured gene counts.

Two tasks were defined using this data. The first, norman-ood, assesses the ability of each model to predict gene expression profiles for held-out cells that have received perturbation combinations that are not included in the training set. Each model was trained on cells that received a single guide, along with [0, 25, 75, 100]% of combinations. Held out cells receiving the final 25% of combinations were used to evaluate each model. This analysis was performed for 5 random splits of the combinations. The second task, norman-data-efficiency, assesses how efficiently the models can learn combination phenotypes when trained on cells that have received a single guide and increasing numbers of cells sampled uniformly across all combinations.

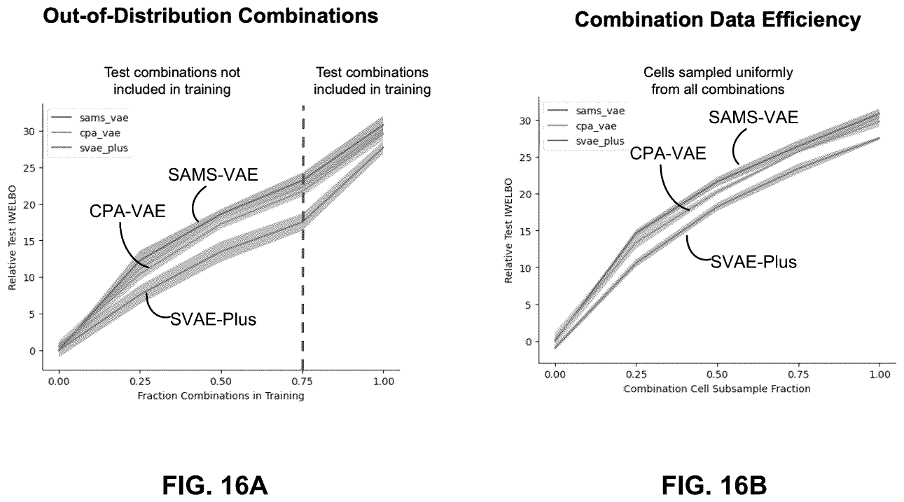

Quantitative results are presented in FIGS. 16A and 16B. Specifically, FIGS. 16A and 16B show test set IWELBO results from norman-ood and norman-data-efficiency experiments. IWELBO values are plotted relative to the mean IWELBO of SAMS-VAE models with 0 combinations for each held-out split. SAMS-VAE and CPA-VAE in this figure both use correlated inference. SAMS-VAE performs the best across both tasks, with CPA-VAE a close second. In norman-ood, SAMS-VAE, CPA-VAE, and SVAE+ all perform very similarly at predicting combination phenotypes when trained with zero combinations. However, SAMS-VAE and CPA-VAE, which both have additive latent mechanisms and can thus share information across individual perturbations and combinations, improve more quickly at predicting held out combination phenotypes than SVAE+, which encodes combinations as entirely new perturbations equivalent to “no treatment”. SVAE+ still improves as more combinations are included in training, which may be attributable to improvements in the encoder and decoder (shared across perturbations). When all combinations are included in the training set, all models improve and the gap between additive latent models and SVAE+ is reduced. In norman-data-efficiency, a similar pattern is observed as SAMS-VAE and CPA-VAE improve more quickly than SVAE+, though the gap is smaller when all combinations are included in the training set.

### Application to Imaging Data

SAMS-VAE is applied to non-genomics datasets perturbation datasets by selecting an appropriate likelihood distribution. As a proof of concept, SAMS-VAE is applied to a cell-painting screening dataset, where perturbations are applied to cells and phenotypes are captured by fluorescence microscopy imaging. Images are obtained from the CPJUMP1 dataset with CRISPR KO perturbations, as described in Chandrasekaran, S. et al., Three million images and morphological profiles of cells treated with matched chemical and genetic perturbations. bioRxiv, 2022, which is hereby incorporated by reference in its entirety. CellProfiler features (as described in McQuin, A. et al., Cellprofiler 3.0: Next-generation image processing for biology. PLoS biology, 16(7):e2005970, 2018) from segmented cells, which capture image derived statistics of cells, are used as observations. After normalization and filtering of highly correlated derived statistics, a gaussian likelihood is used to model the dataset with SAMS-VAE.

### Example 2: Application of SAMS-VAE for Learning Treatment Representations from Pooled Optical Screens

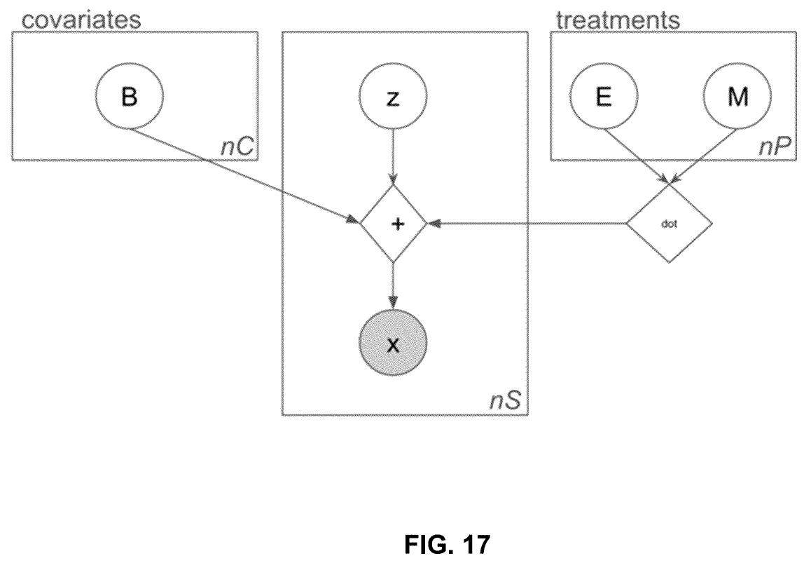

In this example, a modified version of SAMS-VAE was employed that adds a latent variable for covariates along with the treatment and mask embeddings. In case of pooled optical screens, the treatments are gene knockouts and covariates (B) include well location and plate barcode. The modified version of SAMS-VAE is shown in FIG. 17. In particular the modified SAMS-VAE shown in FIG. 17 includes additional covariates (B) in comparison to the model in FIG. 8.

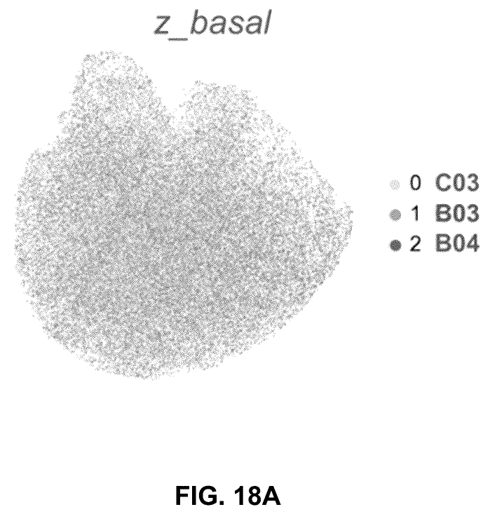

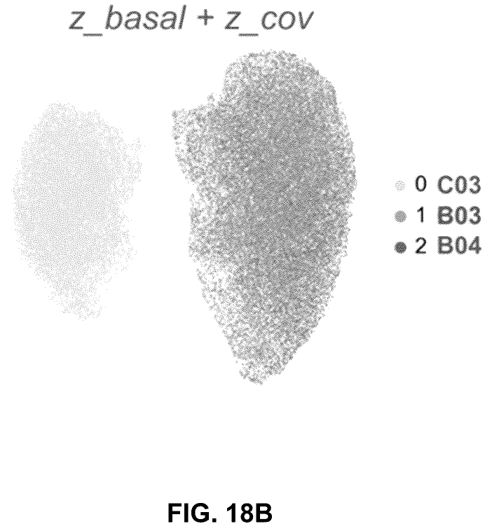

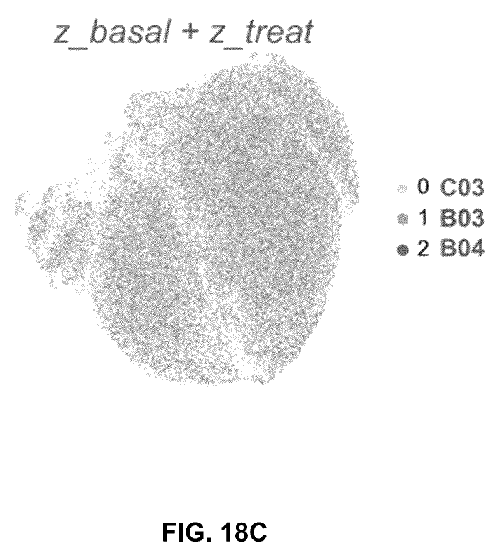

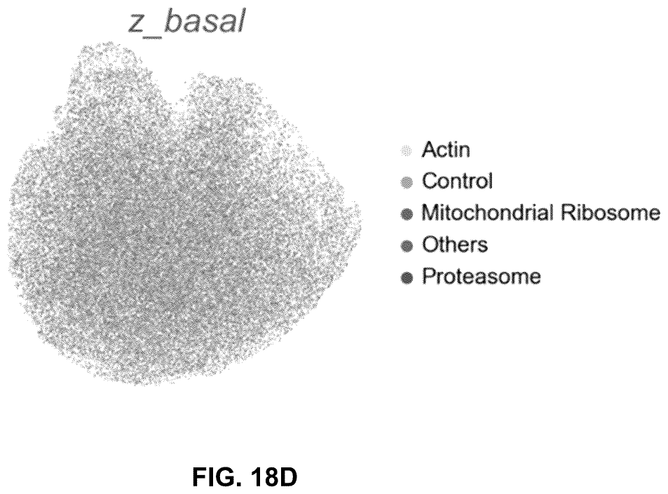

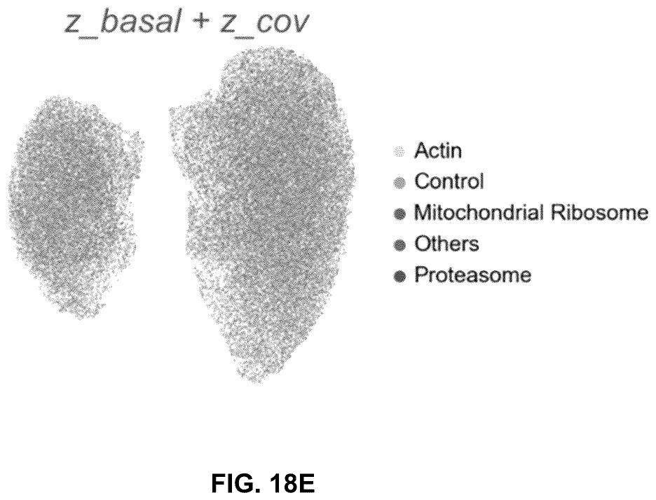

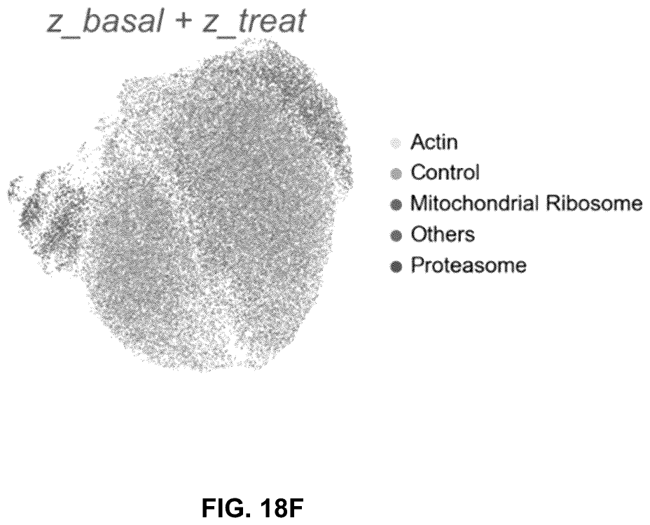

Here, self-supervised image embeddings were used as input to the variational auto encoder and the latent state was decomposed into additive components of z_basal (z), z_covariate (z_B) and a masked z_treatment (z_E·z_M). The model was trained to learn the latent vectors for treatments, basal and covariate states. This experiment was performed on a 124 gene knockout (KO) pooled optical screening (POSH) dataset. FIGS. 18A-18C and FIGS. 18D-18F show the embeddings that compose additively, thereby demonstrating the application of SAMS-VAE for image based pooled screens. For example, FIG. 18A shows embeddings of the z_basal state, FIG. 18B shows composed embeddings of the z_basal and z_covariate states, and FIG. 18C shows composed embeddings of the z_basal and z_treatment states. Similarly, FIG. 18C shows embeddings of the z_basal state, FIG. 18D shows composed embeddings of the z_basal and z_covariate states, and FIG. 18E shows composed embeddings of the z_basal and z_treatment states.

### REFERENCES

Srinivas Niranj Chandrasekaran, Beth A. Cimini, Amy Goodale, Lisa Miller, Maria Kost-Alimova, Nasim Jamali, John Doench, Briana Fritchman, Adam Skepner, Michelle Melanson, John Arevalo, Juan C. Caicedo, Daniel Kuhn, Desiree Hernandez, Jim Berstler, Hamdah Shafqat-Abbasi, David Root, Sussane Swalley, Shantanu Singh, and Anne E. Carpenter. Three million images and morphological profiles of cells treated with matched chemical and genetic perturbations. bioRxiv, 2022. doi: 10.1101/2022.01.05.475090. URL httpsJ/www.biorxiv.org/content/early/2022/01/05/2022.01.05.475090.
Eric Jang, Shixiang Gu, and Ben Poole. Categorical reparameterization with gumbel-softmax, 2017.
Romain Lopez, Jeffrey Regier, Michael B. Cole, Michael I. Jordan, and Nir Yosef. Deep generative modeling for single-cell transcriptomics. Nature Methods, 2018. doi: 10.1038/s41592-018-0229-2.
Romain Lopez, Nataga Tagasovska, Stephen Ra, Kyunghyn Cho, Jonathan K. Pritchard, and Aviv Regev. Learning causal representations of single cells via sparse mechanism shift modeling, 2022. URL https: //arxiv.org/abs/2211.03553.
Mohammad Lotfollahi, Anna Klimovskaia Susmelj, Carlo De Donno, Yuge Ji, Ignacio L. Ibarra, F. Alexander Wolf, Nafissa Yakubova, Fabian J. Theis, and David Lopez-Paz. Learning interpretable cellular responses to complex perturbations in high-throughput screens. bioRxiv, 2021. doi: 10.1101/2021.04.14.439903. URL httpsJ/www.biorxiv.org/content/early/2021/05/18/2021.04.14.439903.
Claire McQuin, Allen Goodman, Vasiliy Chemyshev, Lee Kamentsky, Beth A Cimini, Kyle W Karhohs, Minh Doan, Liya Ding, Susanne M Rafelski, Derek Thirstrup, et al. Cellprofiler 3.0: Next-generation image processing for biology. PLoS biology, 16(7):e2005970, 2018.
Thomas M. Norman, Max A. Horlbeck, Joseph M. Replogle, Alex Y. Ge, Albert Xu, Marco Jost, Luke A. Gilbert, and Jonathan S. Weissman. Exploring genetic interaction manifolds constructed from rich single-cell phenotypes. Science, 365(6455):786-793, 2019. doi: 10.1126/science.aax4438. URL https://www.science.org/doi/abs/10.1126/science.aax4438.
Joseph M. Replogle, Reuben A. Saunders, Angela N. Pogson, Jeffrey A. Hussmann, Alexander Lenail, Alina Guna, Lauren Mascibroda, Eric J. Wagner, Karen Adelman, Gila Lithwick-Yanai, Nika Iremadze, Florian Oberstrass, Doron Lipson, Jessica L. Bonnar, Marco Jost, Thomas M. Norman, and Jonathan S. Weissman. Mapping information-rich genotype-phenotype landscapes with genome-scale perturb-seq. Cell, 185(14):2559-2575.e28, 2022. ISSN 0092-8674. doi: https://doi.org/10.1016/j.cell.2022.05.013. URL https-J/www.sciencedirect.com/science/article/piVS0092867422005979.
Yusuf Roohani, Kexin Huang, and Jure Leskovec. Gears: Predicting transcriptional outcomes of novel multi-gene perturbations. bioRxiv, 2022. doi: 10.1101/2022.07.12.499735.

1. A method of performing modeling of a cellular response to one or more perturbations, the method comprising:
accessing a plurality of disentangled representations in a latent space, the plurality of disentangled representations comprising:
a basal state representation (z^{b}) of a cell;
a learned treatment mask (mₜ) for a perturbation; and
a treatment representation (eₜ) for the perturbation;
generating a sparse treatment representation (z^{p}) representing a combination of the learned treatment mask (mₜ) and the treatment representation (eₜ);
combining the basal state representation (z^{b}) and the sparse treatment representation (z^{p}) to generate a treated representation (z) representing a perturbed cell in the latent space; and
predicting the cellular response to the one or more perturbations using the treated representation (z) representing a perturbed cell in the latent space.

2. The method of claim 1, wherein predicting the cellular response comprises determining a magnitude of a shift in the latent space between the treated representation (z) representing a perturbed cell in the latent space and the basal state representation (z^{b}).

3. The method of claim 1, wherein predicting the cellular response comprises decoding the treated representation (z) in the latent space to predict a phenotypic output of the cell in response to the one or more perturbations.

4. The method of claim 3, wherein the phenotypic output comprises one or more of cell sequencing data, protein expression data, gene expression data, image data, cell metabolic data, cell morphology data, or cell interaction data.

5-7. (canceled)

8. The method of claim 3, wherein decoding the treated representation (z) in the latent space comprises applying a likelihood function.

9. The method of claim 8, wherein the likelihood function comprises a negative binomial likelihood model or a Gaussian likelihood model.

10. The method of claim 8, wherein the likelihood function is parameterized by a learned inversion dispersion parameter θ_{g}.

11. (canceled)

12. The method of claim 1, wherein the basal state representation (z^{b}) of the cell comprises a vector embedding comprising values sampled from a learned distribution.

13. The method of claim 1, wherein the treatment representation (eₜ) for the perturbation comprises a vector embedding comprising values sampled from a learned distribution.

14. The method of claim 1, wherein the learned treatment mask (mₜ) for the perturbation comprises sparse values.

15. The method of claim 14, wherein the sparse values of the learned treatment mask (mₜ) reflect locations in the latent space affected by the perturbation.

16. The method of claim 14, further comprising using the sparse values of the learned treatment mask (mₜ) to identify one or more affected cellular pathways in response to the perturbation.

17. The method of claim 1, wherein the perturbation is one of a chemical agent, molecular intervention, environmental mimic, or genetic editing agent.

18-24. (canceled)

25. The method of claim 1, wherein the plurality of disentangled representations in the latent space are generated by:
training an autoencoder comprising:
an encoder configured to analyze a phenotypic output of a cell to generate one or more corresponding representations in the latent space; and
a decoder configured to analyze a representation in the latent space to generate a predicted phenotypic output.

26. The method of claim 25, wherein training the autoencoder comprises jointly training the encoder and the decoder.

27. The method of claim 25, wherein training the autoencoder further comprises:
disentangling a representation generated by the encoder in the latent space into a basal state representation of a cell and a treatment representation for a perturbation.

28. The method of claim 25, wherein training the autoencoder further comprises:
training a distribution for sampling a learned treatment mask (mₜ) for a perturbation, the learned treatment mask (mₜ) comprising sparse values reflecting affected cellular pathways in the latent space in response to the perturbation.

29-46. (canceled)

47. The method of claim 1, wherein the method is performed in silico.

48. A method of performing modeling of a cellular response to a first perturbation and a second perturbation, the method comprising:
accessing a plurality of disentangled representations in a latent space, the plurality of disentangled representations comprising:
a basal state representation (z^{b}) of a cell;
a first learned treatment mask (mₜ_{₁}) for the first perturbation;
a second learned treatment mask (mₜ_{₂}) for the second perturbation;
a first treatment representation (eₜ_{₁}) for the first perturbation;
a second treatment representation (eₜ_{₂}) for the second perturbation;
generating a first sparse treatment representation (z^{p}^{₁}) representing a combination of the first learned treatment mask (mₜ_{₁}) and the first treatment representation (eₜ_{₁});
generating a second sparse treatment representation (z^{p}^{₂}) representing a combination of the second learned treatment mask (mₜ_{₂}) and the second treatment representation (eₜ_{₂});
combining the basal state representation (z^{b}) and the first sparse treatment representation (z^{p}^{₁}) and the second sparse treatment representation (z^{p}^{₂}) to generate a treated representation (z) representing a perturbed cell in the latent space; and
predicting the cellular response to the perturbation using the treated representation (z) representing a perturbed cell in the latent space.

49. A method of performing a reversion screen, the method comprising:
accessing a plurality of disentangled representations in a latent space, the plurality of disentangled representations comprising:
a basal state representation (z^{b}) of a cell;
a first learned treatment mask (mₜ_{₁}) for a first perturbation;
a second learned treatment mask (mₜ_{₂}) for a second perturbation;
a first treatment representation (eₜ_{₁}) for the first perturbation;
a second treatment representation (eₜ_{₂}) for the second perturbation;
generating a first sparse treatment representation (z^{p}^{₁}) representing a combination of the first learned treatment mask (mₜ_{₁}) and the first treatment representation (eₜ_{₁});
generating a second sparse treatment representation (z^{p}^{₂}) representing a combination of the second learned treatment mask (mₜ_{₂}) and the second treatment representation (eₜ_{₂});
comparing the first sparse treatment representation (z^{p}^{₁}) and the second sparse treatment representation (z^{p}^{₂}) to determine whether effects of the second perturbation counteract effects of the first perturbation.

50-101. (canceled)
